(19) United States
(12) Patent Application Publication
(10) Pub. No.: US 2016/0011207 A1
(43) Pub. Date: Jan. 14, 2016

(54) DIAGNOSIS AND THERAPY OF CHRONIC INFLAMMATION-INDUCED DISORDERS

(71) Applicant: VIRGINIA COMMONWEALTH UNIVERSITY, Richmond, VA (US)

(72) Inventor: Youngman OH, Richmond, VA (US)

(21) Appl. No.: 14/648,802

(22) PCT Filed: Dec. 5, 2013
(86) PCT No.: PCT/US2013/073242

§ 371 Date: Jun. 1, 2015

Related U.S. Application Data
(60) Provisional application No. 61/734,553, filed on Dec. 7, 2012.

(51) Int. Cl. G01N 33/68; C07K 7/06

(52) U.S. Cl. CPC G01N 33/6893; C07K 7/06; G01N 2333/47; G01N 2800/04

(57) ABSTRACT

Methods and compositions for the diagnosis and treatment of chronic inflammation (obesity)-induced disorders such as insulin resistance, diabetes, cancer and various metabolic disorders, are provided. The methods and compositions detect both full-blown disease and early stage disease by detecting proteolysis, by neutrophil proteases, of insulin-like growth factor binding protein-3 (IGFBP3). Levels of proteolytic fragments of IGFBP3 and/or levels of intact IGFPB-3 and/or levels/activity neutrophil proteases are detected. Agents (e.g. peptide agents) that inhibit the proteolysis of IGFPB-3 and methods of using the agents to prevent and treat chronic inflammation (obesity)-induced disorders are also provided.

### BACKGROUND OF THE INVENTION

1. Field of the Invention

The invention generally relates to the diagnosis and treatment of chronic inflammation-induced diseases such as insulin resistance, diabetes, cardiovascular disease, cancer and various metabolic disorders. In particular, the invention provides diagnostic tests for detecting proteolysis, by neutrophil proteases, of insulin-like growth factor binding protein-3 (IGFBP3), as well as peptide agents for inhibiting IGFBP3 proteolysis.

2. Background of the Invention

According to the World Health Organization, obesity has overtaken malnutrition and being underweight as a cause of premature death. Nearly two-thirds of the adults in the United States are overweight or obese. In recent decades, a rapid increase in the prevalence of obesity in adolescents has been observed, predicting an epidemic of metabolic complications in this population. Lifestyle changes to counteract obesity and physical inactivity have been emphasized as the first line of defense against progression to type 2 diabetes mellitus; however, despite an emphasis on lifestyle changes, reductions in obesity rates have not been observed.

Obesity is a complex disorder and is a major risk factor associated with increased risk of insulin resistance, T2DM, cardiovascular disease, hypertension and other metabolic disorders. It is generally established that low-grade chronic inflammation contributes substantially to the burden of obesity.

Colorectal cancer (CRC) is the 3rd most common cause of cancer deaths in the United States. Despite improved radio- and chemo-therapeutic regimens and surgical outcomes, almost half of the colorectal carcinoma patients relapse within 5 years of treatment and inevitably succumb to the disease. One of the paradigm shifts in colorectal cancer research describes that colonic inflammation may contribute to development and progression of colorectal cancer (CRC). Indeed, most CRC and all colitis-associated colon cancer (CAC) have constitutive activation of transcriptional factors that are essential components of multiple inflammatory pathways, namely nuclear factor-κB (NF-κB). Furthermore, the risk of CRC is higher in ulcerative colitis (UC) patients than in the general population and the duration and severity of UC correlate with the risk of developing CAC. These findings suggest a critical role for colonic inflammation for pathogenesis and pathophysiology of CRC.

Neutrophils are the most common leukocyte type in the blood, and play an essential role in innate immune defense against invading pathogens and inflammatory response. With respect to their involvement in CRC it has been reported that an elevated inflammatory neutrophil-to-lymphocyte ratio predicts a significantly higher risk of death in CRC. Furthermore, recent studies demonstrated that depletion of neutrophil in circulation results in significant reduction of the number and size of CRC. These observations suggest that neutrophils play an indispensable role in the initiation and progression of CAC. As discussed above, inflammatory neutrophils produce a number of serine proteinases, such as neutrophil elastase (NE), cathepsin G (CG) and proteinase-3 (PR3). Interestingly, it has been shown that the proteolytic activity of these neutrophil serine proteinases (NSPs) is not necessarily crucial for their antimicrobial activity, but is involved in the pathogenesis of neutrophil-dependent inflammation and progression of chronic inflammatory diseases, in particular development of inflammatory bowel disease (IBD) such as UC and Crohn's disease and further progression of CAC in colon.

Insulin-like growth factor binding protein-3 (IGFBP-3) has been demonstrated to have not only direct antitumor functions in a variety of human cancers but also anti-inflammatory properties in normal and cancer cells through activation of a specific receptor, IGFBP-3R. Recently, the importance of the IGF system in CRC has been addressed in large prospective studies by demonstrating a strong correlation between low IGFBP-3 levels in the circulation and increased cancer risk. A significant reduction of IGFBP-3 has been also observed in chronic inflammatory diseases including IBD. These findings strongly suggest anti-inflammatory and antitumor role of IGFBP-3 in pathogenesis and pathophysiology of colonic inflammation and CAC.

There is a need in the art for additional tools for combating such diseases. For example, it would be of benefit to have available reliable, low cost diagnostic tests for detecting chronic inflammation (obesity) associated diseases, especially in the early stages of disease development. In addition, it would be advantageous to have available new efficacious agents for preventing and/or treating chronic inflammation (obesity) associated diseases.

### SUMMARY OF THE INVENTION

Methods and compositions for the diagnosis and treatment of chronic inflammation (obesity)-induced disorders such as insulin resistance, diabetes, cardiovascular disease and various metabolic disorders as well as cancer, are provided. The methods and compositions detect both full-blown disease and early stage disease by detecting, in a biological sample, the proteolysis by neutrophil proteases of insulin-like growth factor binding protein-3 (IGFBP-3). For example, one or more of levels of proteolytic fragments of IGFBP3, levels of intact IGFPB-3, ratio of IGFBP-3 fragments over total IGFBP-3 (intact+fragments) and levels and/or activity of neutrophil proteases are measured and correlated with suitable reference values in order to determine whether or not the subject being tested has, or is in the course of developing (is at risk of developing) a chronic inflammation (obesity)-related disorder. In addition, agents, including novel peptide agents, that inhibit the proteolysis of IGFPB-3 and methods of preventing and treating chronic inflammation (obesity)-induced disorders are provided.

It is an object of this invention to provide methods for early diagnosis of a subject having a tendency to develop chronic inflammation associated with obesity. The methods comprise 1) contacting a biological sample from the subject with at least one agent which selectively binds to at least one biomarker of insulin-like growth factor-binding protein 3 (IGFBP-3) proteolysis by at least one neutrophil protease, wherein said step of contacting is carried out under conditions which allow the at least one agent to form an agent-biomarker complex with the at least one biomarker to which it selectively binds; 2) detecting a level of agent-biomarker complex in the sample; 3) comparing the level of agent-biomarker complex to at least one pre-determined reference level of agent-biomarker complex, wherein the at least one pre-determined reference level includes a first pre-determined reference level from a control population of individuals who do not have a tendency to develop chronic inflammation associated with obesity, and i) if the level of complex differs from the first pre-determined reference level, then concluding that the subject has a tendency to develop chronic inflammation associated with obesity; and ii) if the level of complex does not differ from the first pre-determined reference level, then concluding that the subject does not have a tendency to develop chronic inflammation associated with obesity. The at least one biomarker may be, for example, one or more proteolytic fragments generated by cleavage of insulin-like growth factor-binding protein 3 (IGFBP-3) by a neutrophil protease; IGFBP-3; at least one neutrophil protease.

In some aspects, the biomarker is IGFBP-3 and a level of IGFBP-3 lower than the first pre-determined reference level, then the subject a practitioner of the method would conclude that the subject does have a tendency to develop chronic inflammation associated with obesity. In this aspect, i) levels of intact IGFBP-3 equal to or greater than 4500 ng/ml are considered normal; ii) levels of intact IGFBP-3 greater than 4100 ng/ml but less than 4500 ng/ml indicate early stage disease; and iii) levels of intact IGFBP-3 less than 4100 ng/ml indicate the presence of disease.

In other aspects, the biomarker is at least one neutrophil protease. In these aspects, differing by being equal to or higher than the first pre-determined reference level is an indication that the subject does have a tendency to develop chronic inflammation associated with obesity. The at least one neutrophil protease may be, for example proteinase 3 (PR3), neutrophil elastase (NE) and cathepsin G (CG).

In yet other aspects, the biomarker is one or more proteolytic fragments. In these aspects, differing by being equal to or higher than the first pre-determined reference level is an indication that the subject does have a tendency to develop chronic inflammation associated with obesity. The one or more proteolytic fragments may be, for example: a 28-kDa fragment formed by proteolysis of IGFBP-3 by PR-3, a 20-kDa fragment formed by proteolysis of IGFBP-3 by PR-3, a 25-kDa fragment by proteolysis of IGFBP-3 by NE, a 28-kDa fragment formed by proteolysis of IGFBP-3 by CG, a 27-kDa fragment formed by proteolysis of IGFBP-3 by CG, a 25-kDa fragment formed by proteolysis of IGFBP-3 by CG, a 20-kDa fragment formed by proteolysis of IGFBP-3 by CG, and a 18-kDa fragment formed by proteolysis of IGFBP-3 by CG.

In some aspects, the chronic inflammation (obesity)-associated disorder is, for example, insulin resistance, type-2 diabetes, cancer (e.g. colon cancer) or a metabolic disorder.

The at least one pre-determined reference level may further include a reference value such as: a reference value from a control population of individuals who are developing an chronic inflammation (obesity)-associated disorder; a reference value from a control population of individuals who have an chronic inflammation (obesity)-associated disorder; and a reference value from the subject prior to receiving therapy to prevent an chronic inflammation (obesity)-associated disorder.

The invention also provides methods of diagnosing, in a subject in need thereof, whether or not the subject has or is developing a chronic inflammation (obesity) associated disorder. The methods comprise the steps of: 1) contacting a biological sample from the subject with at least one agent which selectively binds to at least one biomarker of insulin-like growth factor-binding protein 3 (IGFBP-3) proteolysis by at least one neutrophil protease, wherein said step of contacting is carried out under conditions which allow the at least one agent to form an agent-biomarker complex with the at least one biomarker to which it selectively binds; 2) detecting a level of agent-biomarker complex in the sample; and 3) comparing the level of agent-biomarker complex to at least one pre-determined reference level of agent-biomarker complex, wherein the at least one pre-determined reference level includes a first reference level from a control population of individuals who do not have and are not developing a chronic inflammation (obesity)-associated disorder. If the level of complex differs from the first pre-determined reference level, then a practitioner of the methods concludes that the subject has or is developing a chronic inflammation (obesity)-associated disease or condition; however, ii) if the level of complex is the same as the first pre-determined reference level, then one would conclude that the subject does not have and/or is not developing a chronic inflammation (obesity)-associated disease or condition. The at least one biomarker may be, for example, one or more proteolytic fragments generated by cleavage of insulin-like growth factor-binding protein 3 (IGFBP-3) by a neutrophil protease; IGFBP-3; at least one neutrophil protease.

In some aspects, the biomarker is intact IGFBP-3 and a level of intact IGFBP-3 lower than the first pre-determined reference level, then the subject a practitioner of the method would conclude that the subject does have a tendency to develop chronic inflammation associated with obesity. In this aspect, i) levels of intact IGFBP-3 equal to or greater than 4500 ng/ml are considered normal; ii) levels of intact IGFBP-3 greater than 4100 ng/ml but less than 4500 ng/ml indicate early stage disease; and iii) levels of intact IGFBP-3 less than 4100 ng/ml indicate the presence of disease.

In some aspects, the biomarker is ratio of IGFBP-3 fragments of total IGFBP-3 (intact+fragments) and ratio of IGFBP-3 fragments of total IGFBP-3 higher than the first pre-determined reference level, then the subject a practitioner of the method would conclude that the subject does have a tendency to develop chronic inflammation associated with obesity. In this aspect, i) ratio of IGFBP-3 fragments of total IGFBP-3 equal to or smaller than the first pre-determined reference level is considered normal; ii) ratio of IGFBP-3 fragments greater than 25% but less than 50% compared to the first pre-determined reference level indicates early stage disease; and iii) ratio of IGFBP-3 fragments greater than 50% compared to the first pre-determined reference level indicates the presence of disease.

In other aspects, the biomarker is at least one neutrophil protease. In these aspects, differing by being equal to or higher than the first pre-determined reference level is an indication that the subject does have a tendency to develop chronic inflammation associated with obesity. The at least one neutrophil protease may be, for example proteinase 3 (PR3), neutrophil elastase (NE) and cathepsin G (CG).

In yet other aspects, the biomarker is one or more proteolytic fragments. In these aspects, differing by being equal to or higher than the first pre-determined reference level is an indication that the subject does have a tendency to develop chronic inflammation associated with obesity. The one or more proteolytic fragments may be, for example: a 28-kDa fragment formed by proteolysis of IGFBP-3 by PR-3, a 20-kDa fragment formed by proteolysis of IGFBP-3 by PR-3, a 25-kDa fragment by proteolysis of IGFBP-3 by NE, a 28-kDa fragment formed by proteolysis of IGFBP-3 by CG, a 27-kDa fragment formed by proteolysis of IGFBP-3 by CG, a 25-kDa fragment formed by proteolysis of IGFBP-3 by CG, a 20-kDa fragment formed by proteolysis of IGFBP-3 by CG, and a 18-kDa fragment formed by proteolysis of IGFBP-3 by CG.

In some aspects, the chronic inflammation (obesity)-associated disorder is, for example, insulin resistance, type-2 diabetes, cancer (e.g. colon cancer) or a metabolic disorder.

The at least one pre-determined reference level may include a reference value such as, for example: a reference value from a control population of individuals who are developing an chronic inflammation (obesity)-associated disorder; a reference value from a control population of individuals who have an chronic inflammation (obesity)-associated disorder; a reference value from a control population of individuals who are receiving therapy to prevent an chronic inflammation (obesity)-associated disorder; a reference value from a control population of individuals who are receiving therapy to treat an chronic inflammation (obesity)-associated disorder; a reference value from the subject prior to receiving therapy to prevent an chronic inflammation (obesity)-associated disorder; a reference value from the subject prior during therapy to prevent an chronic inflammation (obesity)-associated disorder; a reference value from the subject prior to receiving therapy to prevent an chronic inflammation (obesity)-associated disorder; and a reference value from the subject during therapy to treat an chronic inflammation (obesity)-associated disorder.

The invention also provides methods of preventing or treating a chronic inflammation (obesity)-associated disorder in a subject in need thereof, comprising administering to the subject a therapeutically effective amount of one or more inhibitors that inhibit proteolysis of IGFBP-3. In some aspects, the one or more inhibitors include at least one of: an inhibitor of proteinase 3 (PR3), an inhibitor of neutrophil elastase (NE), and an inhibitor of cathepsin G (CG). In other aspects, the inhibitor of PR3 is a peptide having the amino acid sequence: LIRCAML (SEQ ID NO: 1), or derivatives or mimetics thereof.

The invention further provides an isolated peptide having the amino acid sequence LIRCAML (SEQ ID NO: 1), or an amino acid sequence that is at least 95% identical to LIRCAML (SEQ ID NO: 1).

### BRIEF DESCRIPTION OF THE DRAWINGS

FIG. 1 is a scheme which shows chronic inflammation (obesity)-induced PR3 proteolyzes insulin-like growth factor binding protein-3 (IGFBP3), thereby inhibiting insulin sensitizing IGFBP3/GFBP3R signaling in insulin target tissues such as visceral fat, muscle, and liver.

FIG. 2. Time-dependent IGFBP-3 proteolysis by PR3. Recombinant IGFBP-3 (100 ng) was proteolyzed by treatment with physiological concentrations of PR3 (10 nm) in a time dependent manner.

FIG. 3A-D. IGFBP-3 proteolyzed fragments and PR3 are increased in insulin resistant obese population. A positive correlation exists between IGFBP-3 proteolysis and PR3 (A), whereas an inverse correlation exists between whole body insulin sensitivity index and IGFBP-3 proteolysis (B); as well as PR3 levels (C). n=9. D, representative Western immunoblot analysis of IGFBP-3 proteolysis and PR3 levels in insulin-sensitive (high whole-body insulin sensitivity index, “WBISI”) and insulin-resistant population (WBISI<2.0).

FIG. 4A-B. A, increased serum PR3 and IGFPR-3 proteolysis in high fat diet (HFD) mice. The increased IGFBP-3 proteolytic fragments and PR3 were detected in HFD compared with chow diet (CD) fed mice by Western immunoblotting using mouse IGFBP-3 or PR3 specific antibodies. B, IGFBP-3 proteolysis by PR-3 was inhibited by PR-3 inhibitor, α-1-antitrypsin (AAT). IGFBP-3 proteolytic fragments were detected in recombinant IGFBP-3 (100 ng) and PR3 (500 ng) treated samples by Western immunoblotting. Few IGFBP-3 proteolytic fragments were detected in AAT treated samples.

FIG. 5A-D. Correlation among IFGBP-3 fragments, PR3 and insulin resistance: relationship between total IGFPB-3 fragments and A, PR3 (r=0.837 m p<0.001), B, homeostasis model assessment-estimated insulin resistance (HOMA-IR) (r=0.457, p<0.01), C, body mass index (BMI) (r=0.725, p<0.001) and D, waist circumference (r=0.566, p<0.001) were calculated by Pearson's correlation coefficient.

FIG. 6A-D. A, the increases IGFBP-3 proteolytic fragments and NSPs were detected in serum of overweight and obese groups compared with lean groups. 2 μl of serum were subjected to 10% Western immunoblotting and IGFBP-3, PR3, NE and CG were detected using human specific antibodies. B, In vitro IGFBP-3 proteolysis by PR3, NE and CG. Recombinant IGFBP-3 (100 ng) was proteolyzed by treatment with PR3 (100 nM, 250 nM, and 500 nM), and NE (100 nM and 250 nM) and CG (100 nM, 250 nM) for 20 min. at 37° C. The treated samples were subject to Western immunoblotting using polyclonal IGFBP-3 antibodies. Treatment with PR3 resulted in a significant reduction of intact IGFBP-3 while increasing 28 kDa and 20 kDa fragments at eth concentration of 100 nm (lane 3) and the effect was more marked at higher concentrations of 250 and 500 nM (lanes 4 and 5). NE and CG proteolyze IGFBP-3 into a major 25 kDa fragment at concentrations of 100 and 250 nM (lanes 6-9); C, the amount of total IGFBP-3 fragments normalized to BMI 20 was significantly higher in overweight and obese groups. D, The amount of PR3 expression normalized to BMI 20 was significantly higher in overweight and obese groups. BMI (kg/m2): lean (18.5-24.9), overweight (25-29.9), and obese (>30). Lean n=15; overweight n=17, and obese n=6. *p<0.05, **p<0.01, ***p<0.0001.

FIG. 7A-D. Threshold of intact IGFBP-3 concentration in lean and obese individuals. A. The amount of intact-IGFBP-3 measured by ELISA in lean group (4600±128.2 ng/ml) was significantly higher than that in overweight (4166±150.8 ng/ml) and obese groups (3909±176.8 ng/ml). p<0.05; B. Correlation between the amount of intact IGFBP-3 and the ratio of intact IGFBP-3 and total IGFBP-3 (r=0.4776, p<0.05) were calculated by Pearson's correlation coefficient. C. IGFBP-3 proteolysis in lean group (0.44±0.03) was significantly lower than that in overweight (0.60±0.04) and obese (0.69±0.04) groups. p<0.01. D. Correlation between the amount of intact IGFBP-3 and IGFBP-3 proteolysis (r=0.4776, p<0.05) were calculated by Pearson's correlation coefficient.

FIG. 8. IGFBP-3 proteolysis of PR-3 was inhibited by PR3 inhibitor α-1-antitrypsin (AAT) and the small peptide inhibitor (LIRCAML, SEQ ID NO: 1) in a dose-dependent manner. Recombinant IGFBP-3 (100 ng) was proteolyzed by treatment with 100 nm PR3 for 60 min. at 37° C. (lane 2). Proteolysis was inhibited by treatment with AAT (lane 8). The small peptide inhibitor of NSPs inhibited IGFBP-3 proteolysis in a dose-dependent manner (0.5-20 μM). Treatment with the peptide a concentration of 0.5 μM resulted in more than 50% inhibition (not shown). The inhibitory effect of the peptide at a concentration of 20 μM was comparable to that of 100 nM AAT (lanes 7 and 8).

FIG. 9A-C. Inhibitory effect of the small peptide on NSP-induced IGFBP-3 proteolysis. A. IGFBP-3 proteolysis of PR3 was inhibited by PR3 inhibitor α-1-antitrypsin (AAT) and the small peptide inhibitor (LIRCAML, SEQ ID NO: 1) in a dose-dependent manner. Recombinant IGFBP-3 (100 ng) was proteolyzed by treatment with 100 nM PR3 for 60 min at 37° C. (lane 3). Proteolysis was inhibited by treatment with AAT (lane 1). The small peptide inhibitor of NSPs inhibited IGFBP-3 proteolysis in a does-dependent manner (0.05-1 μM) (lane 4-8). B. In vitro IGFBP-3 proteolysis by NE and CG. Proteolysis was inhibited by treatment with AAT and the small peptide inhibitor (L-I-R-C-A-M-L, SEQ ID NO: 1). Recombinant IGFBP-3 (100 ng) was proteolyzed by treatment with NE (70 nM) and CG (200 nM) for 20 min. at 37° C. The treated samples were subject to Western immunoblotting using polyclonal IGFBP-3 antibodies. NE and CG proteolyze IGFBP-3 (lanes 2 and 7). The small peptide inhibitor of NSPs inhibited IGFBP-3 proteolysis by NE or CG in a dose-dependent manner (0.5-1 μM). C. The sequence of small inhibitory peptide (LIRCAML). Putative small inhibitory peptide binding site on PR3 (NTGSSFVI) and NE (NTGSSFVR), of which sequences are well conserved in both PR3 and NE.

FIG. 10. Insulin tolerance test (ITT). After 6 hours fasting, ITT was done by injecting 0.75 unit/kg insulin intraperitoneally into HFD C57BL/6 mice treated with 60 mg/kg AAT or PBS for 7 weeks and measuring blood glucose levels at the indicated time points. Blood glucose levels in HFD C57BL6 mice treated with 60 mg/kg AAT for 7 weeks were significantly lower than those in control HFD C57BL6 mice treated with PBS. (15 min, 60 min, 90 min and 120 min after insulin injection, p<0.05), suggesting that AAT normalizes blood glucose levels in HFD C57BL6 mice by enhancing insulin sensitivity.

FIGS. 11 A and B. The IGFBP-3/IGFBP-3R system in colon cancer cells. A, basal expression of IGFBP-3 mRNA (top) and IGFBP-3R mRNA (bottom) Analyzed by quantitive RT-PCR and Western immunoblotting, respectively. Mean+/−SD, n=3 in duplicate. B, IGFBP-3-induced apoptosis in HT-29 cells (top). Cell death assay was performed 2 days after infection. Apototic death was measured using Cell Death Detecting ELISA. Representative immunoblot analysis of Ad:IGFBP-3 (Bottom). AdEV: adenoviral plasmids with empty vector; Ad:IGFBP-3: with IGFBP-3 cDNA; Ad:IGFBP-3GGG: with IGFBP-3GGG mutant cDNA.

### DETAILED DESCRIPTION

Chronic inflammation (obesity)-induced activation of inflammatory neutrophil serine proteases causes proteolysis of anti-inflammatory, insulin-sensitizing IGFBP-3 in the circulation of obese/overweight individuals. As a result, without suitable intervention, the individual eventually develops insulin resistance and one or more of the diseases/conditions associated with insulin resistance. Therefore, serum neutrophil serine proteases and IGFBP-3 proteolysis are excellent markers for monitoring progression towards chronic inflammation (obesity)-induced insulin resistance, and are targets for therapeutic prevention and treatment of chronic inflammation (obesity) related diseases.

Accordingly, the invention provides methods and compositions for the diagnosis, prevention and treatment of obesity-induced chronic inflammation disorders, e.g. insulin resistance, diabetes, cardiovascular diseases, and various metabolic disorders, and others as described further below. The diagnostic methods, described in detail below, generally involve detecting, in a biological sample from a subject, the proteolysis of insulin-like growth factor binding protein-3 (IGFBP-3) by one or more neutrophil proteases. Exemplary aspects of the invention include measuring one or more of: levels of proteolytic fragments of IGFBP3, levels of intact (non-proteolyzed) IGFPB-3 (IGFPB-3 that has not been cleaved by a neutrophil protease), and levels and/or activity of neutrophil proteases, especially neutrophil proteases for which IGFBP-3 is a substrate. The measured values are correlated with suitable reference values in order to determine whether or not the subject being tested has, or is in the course of developing (e.g. is at risk of developing) a chronic inflammation (obesity)-related disorder. IGFBP3, neutrophil proteases, and proteolytic fragments of IGFBP3 formed by the action of the neutrophil proteases on IGFBP3 thus are “biological markers” of chronic inflammation (obesity)-related conditions/diseases. Advantageously, the methods and compositions of the invention, in addition to detecting the presence of such diseases, also are used to detect very early stages of disease in a subject, e.g. even before more overt disease symptoms appear. This early detection makes it possible to intervene with suitable treatments early on in the progression of the disease, to prevent or avoid the devastating effects of full-blown disease.

Significantly, further aspects of the invention provide agents and methods of their use (e.g. peptide agents and other neutrophil protease inhibitors) that inhibit the proteolysis of IGFPB-3, and which thus can be used to prevent the onset of or treat symptoms of chronic inflammation (obesity) induced disorders.

### Diagnostics

In some aspects, the invention provides diagnostic assays (tests) for diagnosing the presence of one or more chronic inflammation (obesity)-related disorders (which may also be referred to herein as “conditions” or “diseases” that are “chronic inflammation-associated” or “caused by chronic inflammation”, etc.) in a subject. The chronic inflammation may be caused by (linked to, associated with, etc.) excess weight (the subject is “overweight”, or to obesity of the subject. In other aspects, the invention provides diagnostic assays for assessing (determining) the likelihood of a subject to develop a chronic inflammation (obesity)-related disorder, and/or for staging or categorizing the stage of development (progression) of a chronic inflammation (obesity)-related disease in the subject. In particular, the diagnostic assays described herein are useful for the early detection of biomarkers that are the harbingers or predictors of such disorders. “Early” detection can occur, for example, prior to the onset of more readily detectable symptoms (e.g. elevated blood sugar levels, glucose tolerance, etc.), or more overt symptoms such as fatigue, increased thirst, blurred vision, hunger, frequent urination, weight loss, etc.). Early detection includes detection, before a diagnosis of diabetes, when a patent is prediabetic or is trending toward insulin resistance and/or prediabetes. Alternatively, or in addition, the diagnostic methods provided herein may be used in conjunction with other tests (e.g. blood sugar levels, etc.) in order to confirm a diagnosis or disease, or disease risk, or early disease development, etc.

Early detection is achieved by analysis of the “biological makers” that are detected in the sample which are one or more of: levels of intact IGFBP3; levels of neutrophil proteases and/or their activity; and proteolytic fragments of IGFBP3. “Level” generally refers to the amount or concentration (e.g. molar concentration or weight/volume, etc.) of the substance, but may also refer to relative amounts, ratios, etc. of the substance of interest. Quantitation of levels may be direct (e.g. measurement of the number or concentration of molecules) or indirect (e.g. measurement of an activity characteristic of the substance, e.g. enzymatic or other activity).

Exemplary methods for detecting and/or quantifying (measuring) levels of biological marker proteins or peptides (such as the IGFBP3 fragment(s) described herein) in a biological sample involve obtaining a biological tissue sample and contacting the biological sample with a compound or a detection agent capable of detecting the fragment. Generally, such detection and/or quantification assays may involve preparing the sample for reaction with the agent. For example, a reaction mixture may be formed that contains the sample or a portion of the sample, and the agent. The sample and the agent are in contact under appropriate conditions and for a time sufficient to allow the agent to interact with marker that is present in the sample. Frequently, the interaction is a binding interaction, i.e. the detection agent binds to the marker, forming a complex that can be detected within the reaction mixture and/or removed from the reaction mixture and then detected.

As used herein, a “biological sample” may be, for example, blood, plasma, urine, sweat, any tissue (e.g. biopsy tissue of a tissue or organ), saliva, mucous, etc.

As used herein, the term “detection agent” refers to any molecule which is capable of selectively binding to a specifically intended target molecule, such as the proteolytic protein fragments/biological markers described herein. Detection agents can be either synthesized in vitro (e.g. chemically synthesized) or in vivo, i.e. derived from appropriate biological preparations (e.g. in a laboratory animal or from a natural source such as plasma, etc.). For purposes of detection of the target molecule, detection agents may be specifically designed to be labeled with one or more detectable labels, as described herein. Examples of molecules that can be utilized as detection agents to detect the biological markers of the invention include, but are not limited to proteins, peptides, antibodies, organic molecules, etc. The detection agent is frequently an antibody, e.g. a polyclonal or usually a monoclonal antibody specific for reacting with (binding to) a fragment of interest.

Detection and/or quantification assays of a biological marker can be conducted in a variety of ways. For example, one method to conduct such an assay involves anchoring the detection agent onto a solid phase support, also referred to as a substrate; exposing the anchored detection agent to a biological sample or portion of a biological sample that may contain the marker of interest, under conditions and for a period of time sufficient to allow interaction (e.g. binding) between the detection agent and the marker so as to anchor the marker on the support via the detection agent. After the reaction is complete, uncomplexed components may be removed (e.g., by washing) under conditions such that any complexes formed will remain immobilized upon the solid phase.

There are many established methods for anchoring assay components to a solid phase. These include, without limitation, through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored. Other suitable carriers or solid phase supports for such assays include any material capable of binding the class of molecule to which the marker or detection agent belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

The detection of marker/detection agent complexes anchored to the solid phase can be accomplished in a number of methods. In an exemplary embodiment, the detection agent is labeled, either directly or indirectly, with a detectable label, for the purpose of detection and readout of the assay.

Alternatively, in another embodiment, analogous diagnostic and prognostic assays can be conducted with marker and detection agent as solutes in a liquid phase. In such an assay, the complexed marker and detection agent are separated from uncomplexed components by any of a number of techniques, including but not limited to, differential centrifugation, chromatography, electrophoresis and immunoprecipitation. In differential centrifugation, marker/detection agent complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; is Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, complexes are separated from uncomplexed assay components based on size and/or charge, for example. In order to maintain the binding interaction during the electrophoretic process, non-denaturing gel matrix materials and conditions in the absence of reducing agent are typically preferred. SELDI-TOF technique may also be employed on matrix or beads coupled with active surface, or not, or antibody coated surface, or beads.

The “biological makers” that are detected in the sample are generally proteolytic fragments of IGFBP3, or intact unpretolyzed IGFBP3, or even neutrophil proteases. In some aspects, these markers are not labeled prior to separation from other compounds in the sample. For example, sample components may be separated e.g. by size, charge, etc. as described above, and the separated components may then be assayed to identify which, if any, are the targeted proteolytic fragments. Exemplary separation techniques include but are not limited to electrophoresis (where unlabeled samples may be electrophoresed through a matrix (e.g., gel) and bands representing individual, separated components of the sample are detected by staining the gel; or the separated components may be removed from the gel and the composition thereof determined by any suitable method, e.g. sequencing, detection with a specific binding agent, etc.); or by gel chromatography (where unlabeled samples are applied to a column and individual separated protein-containing fractions are collected, e.g. by monitoring wavelength of effluent from the column, and the compositions of the fractions are determined by any suitable method (e.g. sequencing, detection by a detection agent such as an antibody, etc.) in order to determine which, if any, of the fractions contain at least one biomarker of interest

The term “labeled”, with regard to the binding agent (e.g. an antibody) is intended to encompass direct labeling of the binding agent by coupling (i.e., physically linking) a detectable substance to the binding agent, as well as indirect labeling of the binding agent by reactivity with another reagent that is directly labeled. Examples of indirect labeling include detection of a primary antibody using a fluorescently labeled secondary antibody. Exemplary detectable labels include but are not limited to radioactive labels, fluorescent labels, various colorimetric labels, enzyme labels, etc. Multiple labeling can also be performed simultaneously, e.g. in embodiments wherein more than one (a plurality of) fragment-specific antibodies are used, each of which is specific for a different fragment, for the purpose of simultaneously quantifying more than one fragment in a mixture of fragments.

Exemplary in vitro techniques for detection of a biological marker protein or fragment thereof include enzyme linked immunosorbent assays (ELISAs), Western blots, immunoprecipitations, immunofluorescence, immunostaining, immunoelectrophoresis, immunoblotting, Western blotting, various enzyme assays, and the like. Furthermore, in vivo techniques for detection of a marker protein or fragment include introducing into a subject a labeled antibody directed against the protein or fragment thereof. For example, the antibody can be labeled with a radioactive marker whose presence and location in a subject can be detected by standard imaging techniques.

It is also possible to directly detect marker/detection agent complex formation without further manipulation or labeling of either component (marker or detection agent), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

Determination of the ability of a detection agent to recognize a marker can also be accomplished without labeling either assay component (detection agent or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In addition, mass spectrometry (MS) such as liquid chromatography-mass spectrometry (LC-MS, or alternatively HPLC-MS) techniques may also be employed to detect and quantify the proteolytic fragments of interest.

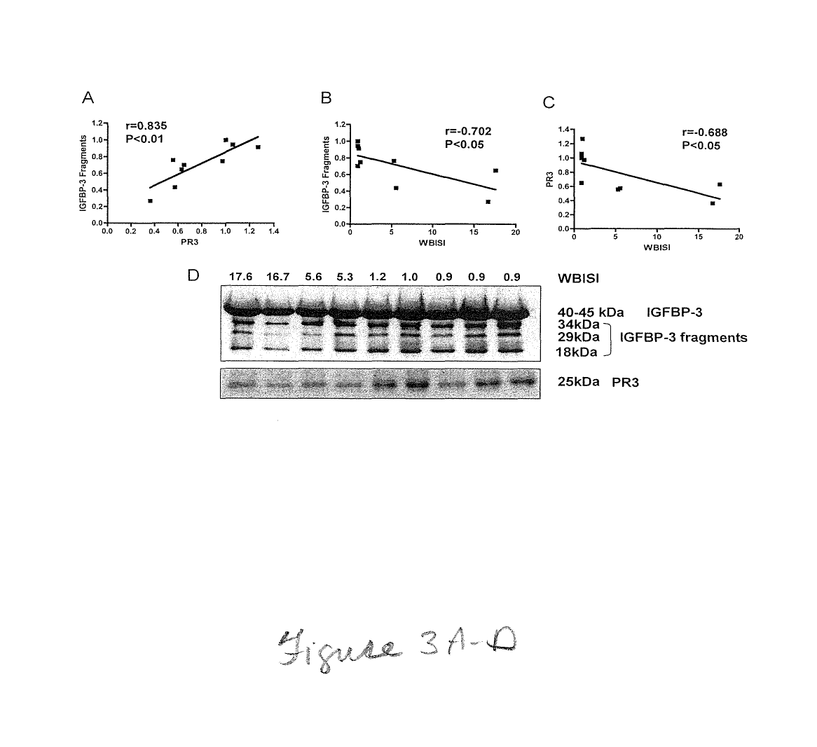

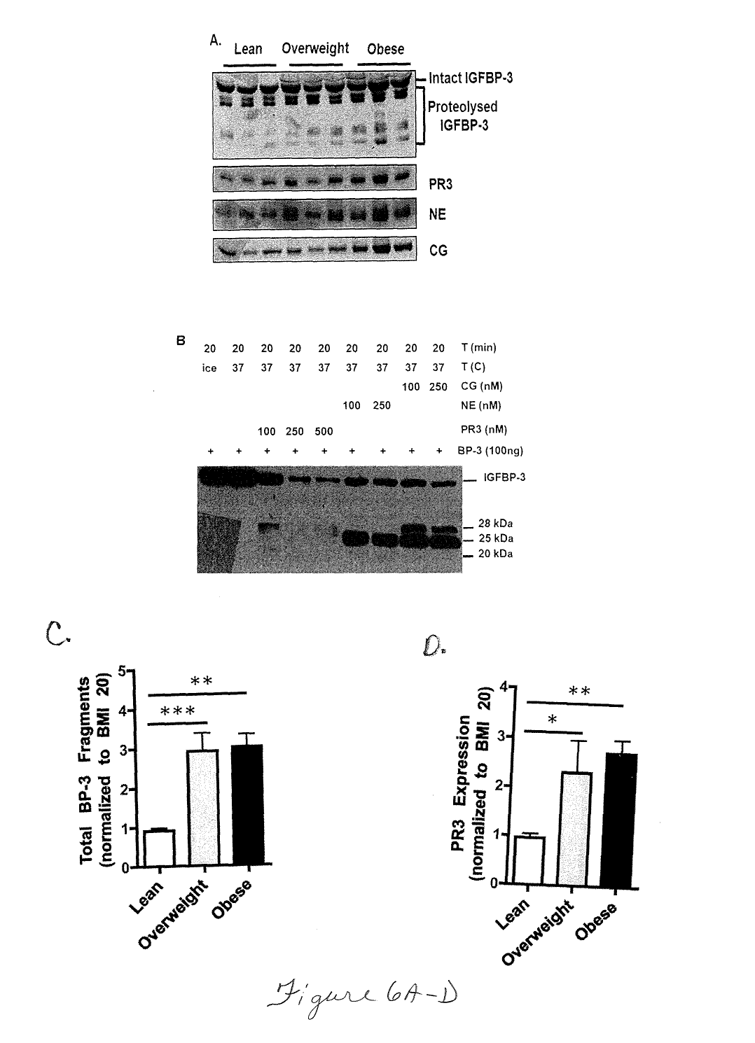

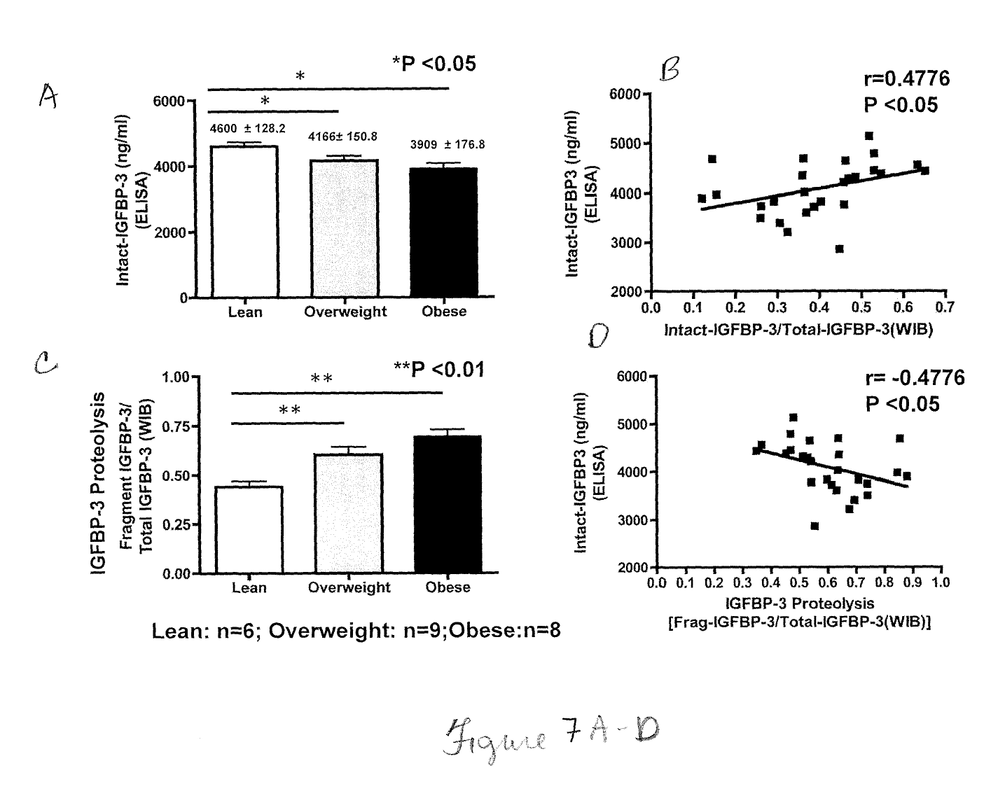

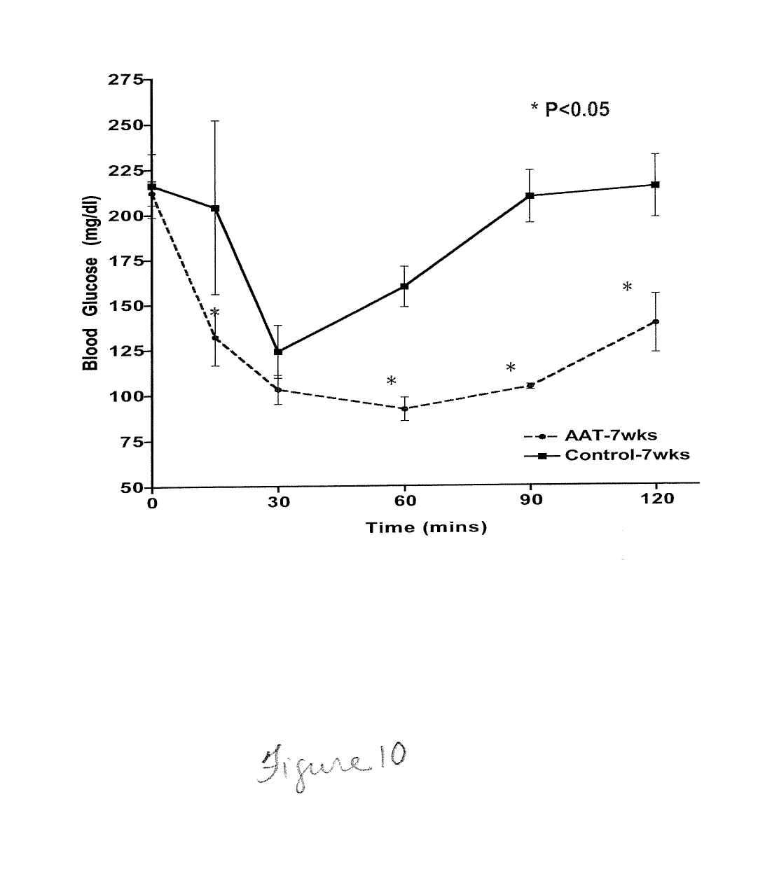

In one aspect, the biomarkers that are detected are one or more proteolytic fragments of IGFBP3. In particular, proteolytic fragments formed by proteolysis of IGFBP3 by neutrophil proteases may be detected. Exemplary neutrophil proteases which are known to digest IGFBP3 include but are not limited to proteinase 3 (PR3), neutrophil elastase (NE), and cathepsin G (CG). In some aspects, proteolytic fragments generated by cleavage of IGFBP3 by PR-3 are detected, namely one or more of the 28 and 20 kDa fragments (as depicted in FIGS. 2,4 and 6). In other aspects, proteolytic fragments generated by cleavage of IGFBP3 by NE are detected, namely one or more of the 25 kDa fragments (as depicted in FIGS. 6 and 9). In yet other aspects, proteolytic fragments generated by cleavage of IGFBP3 by CG are detected, namely one or more of the 27, 25, 18 and 15 kDa fragments (as depicted in FIGS. 6 and 9).

In order to determine the absolute amount of biomarker present in the sample, the results are compared to results obtained using known quantities of suitable reference or control values (e.g. “standards”), e.g. of synthetic or otherwise known purified samples of the biomarker. Alternatively, or in addition, the relative amount of biomarker is then compared to a reference value or values from control populations, in order to determine (draw a conclusion concerning) a diagnosis. Generally, such control values are developed by measuring the level of biomarker in one or more suitable control populations using e.g. a method such as those described above. Control populations generally include a group of healthy subjects who do not have a chronic inflammation (obesity) associated disorder and/or a group of subjects who are known to have a chronic inflammation (obesity) associated disorder, and/or subjects with intermediate stages of such disorders, and/or subjects being treated for such disorders, etc. depending on the nature of the assay, or the reason for conducting the assay. The suitability of such subjects for inclusion in a reference pool may be determined using the present test and/or other tests (e.g. biopsy, measurement of other markers, etc.) and then by calculating statistically relevant control or “cut-off” reference values of the biomarker, or ranges of such values, from the pool of values obtained for a given population.

In general, a result is considered to be positive, i.e. indicative of or consistent with the presence of an chronic inflammation (obesity)-related disorder, or of being at risk of developing an chronic inflammation (obesity) associated disorder, if the level of proteolytic fragments or neutrophil proteases is more than about 10, 20, 30, 40, 50, 60, 70, 80 90, or even 100%, more than that of normal or control subjects. A result is considered to be positive the level of IGFBP3 is less than about 60, 50, 40, 30, 20, 10% (or even lower) than that of normal or control subjects. Increases/decreases are considered to be significant if they statistically significant relative to an appropriate control. Increases/decreases are considered to be significant if they statistically significant relative to an appropriate control.

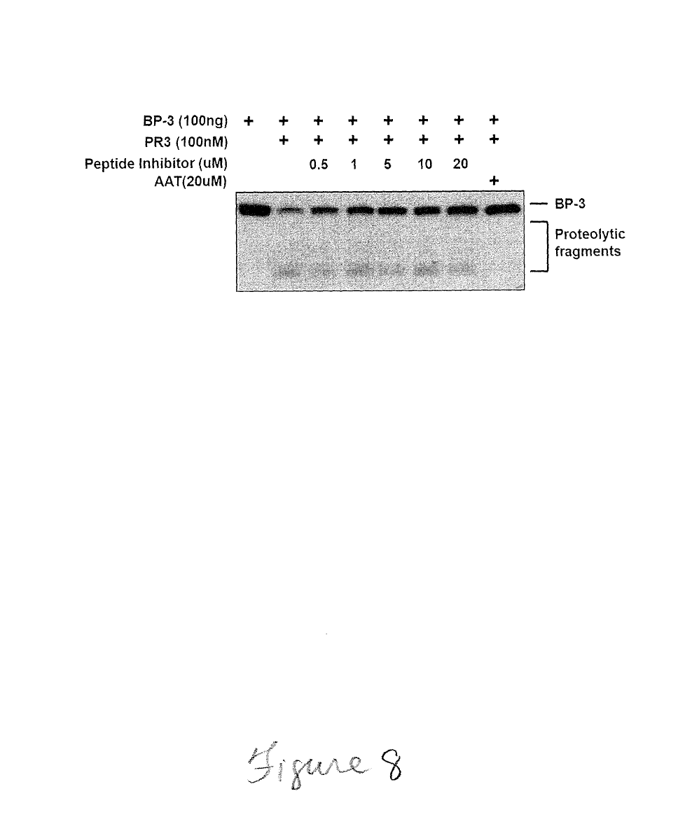

In other aspects, threshold or cut-off values of the biomarkers are provided such that, for intact IGFBP-3 concentrations in the presence of IGFBP-3 protease activity, values that are below the threshold value are considered positive with respect to the presence of disease, whereas values that are equal to or high than the threshold value are considered negative (i.e. are an indicator that disease is not present). Exemplary threshold values are 4100 ng/ml (see, for example, FIG. 7). Those of skill in the art will recognize that threshold values may differ somewhat, depending on gender, age, ethnicity, general health, etc. and can be established by interrogating (sampling) suitable control populations.

However, such higher values may be used as indicators of a pre-disease stage, e.g. of earlier stages in the development of the disease. These nuanced results may thus advantageously suggest the presence of early stage (e.g. “borderline”) or intermediate stages of disease, or of partial control of disease by a therapeutic regimen. In this case, intact IGFBP-3 concentration ranging from 4100 to 4500 ng/ml may be considered to indicate a pre-disease stage (FIG. 7). As above, those of skill in the art will recognize that threshold values may differ somewhat, depending on gender, age, ethnicity, general health, etc. and can be established by interrogating (sampling) suitable control populations.

Any measurable increase in detectable levels of proteolytic fragments is a positive indication that disease or a pre-disease condition is present. Alternatively, or in addition, an increase of about 300% in IGFBP-3 fragments in typically present in both overweight and obese subjects, compared to normal (lean) control subjects (FIG. 7). If concentrations of serum NE are measured, a normal range is considered to be <250 ng/ml whereas the range for an obese subject is >250 ng/ml).

Many types of individuals will benefit from the type of assessments described herein. For example, the subject may be a person who already has symptoms of a an chronic inflammation (obesity)-related disorder, or is already known to have such a disorder, or is already being treated for such a disorder, or is at risk of having or developing such a disorder (e.g. an obese person, a person with a genetic predisposition to such disorders, etc.). Alternatively, the present test may be incorporated into routine blood tests of individuals who have no signs, symptoms, or immediate likely risk of the disease, e.g. the test may be part of a routine blood panel assessment such as those that are frequently prescribed by physicians (e.g. on a yearly basis) for those who are or appear healthy. In this case, the test may be considered a prophylactic or precautionary measure. The test may be part of such an assessment for particular populations, e.g. “senior citizens”, persons with sedentary lifestyles, individuals with hereditary dispositions toward chronic inflammation (obesity), persons taking drugs which cause or contribute to chronic inflammation (obesity), or any other population that might benefit from diagnosis and/or monitoring of lipid metabolism. In addition, while the test subject is usually a human, this is not always the case; veterinary applications of this technology are also contemplated. For example, companion pets may be tested or treated (e.g. cats, dogs, etc.) as may various domesticated animals such as horses, cattle, livestock of any type (sheep, goats, etc.); prize winning or highly trained animals; animals domiciled in protected environments such as zoos, parks, refuges, etc.; rare animals under threat of extinction, and others. Any subject for which the knowledge provided by the tests could be beneficial may be tested and treated as described herein.

The results of the test may be used in conjunction with other diagnostic methods such as e.g. measurement of blood sugar levels, glucose tolerance, etc. The results of the test may also be used in the development and/or adjustment of treatment protocols for patients. Further, once the patient is undergoing treatment or if the patient is already undergoing treatment, the tests and methods described herein may be used to monitor the success or failure (e.g. efficacy) of the treatment, and/or to monitor compliance with the treatment. For example, a patient for whom increased exercise and diet changes have been recommended may be monitored by measuring the level of biomarker in a biological sample at various time intervals (e.g. daily, weekly monthly, etc.) to ascertain whether or not the changes that have been made are sufficient, whether the treatment is efficacious, or whether additional measures should be prescribed. In such cases, the reference values that are used to assess the test results may include one or more biomarker levels previously measured in samples from the patient him- or herself, e.g. samples taken from before treatment, or earlier in treatment, etc. For example, the course of treatment with an agent can be followed, e.g. where measurements are made before and after administration, and the effect of administration can thus be assessed, and further monitored on an ongoing basis, as necessary or advisable.

Types of diseases/conditions that can be detected using the tests include that that are listed below as diseases/conditions that are targeted for treatment.

### Treatments

Other aspects of the invention include methods for treating (preventing, ameliorating, etc.) a disease (disorder, condition, etc.) caused by, associated with or related to obesity-linked chronic inflammation. The methods comprise a step of administering to a subject in need thereof a therapeutically effective amount of at least one of 1) an IGFBP-3 proteolysis inhibitor, e.g. an administering an inhibitor of a protease that is known to digest IGFPB-3 such as PR3, NE, CG, etc.; and 2) administering IGFPB-3. A therapeutic amount is an amount that may entirely eliminate symptoms of the disease or condition being treated, or may ameliorate or lessen such symptoms, or slow the development of the disease or condition.

In one aspect, the agent is an agent that inhibits the activity of at least one neutrophil protease that proteolyzes IGFBP-3. An inhibitor may be a peptide or peptide derivative or peptide mimetic that inhibits, e.g. PR3. An exemplary peptide that may be used in this manner is: LIRCAML (SEQ ID NO: 1), which has an amino acid sequence as represented by SEQ ID NO: 1, or modified derivatives or mimetics thereof. In other aspects, the inhibitors include but are not limited to: ONO-5046, ONO-6818, MR-889, L-694,458, CE-1037, DMP-777, GW-311616, TEI-8362, sivelestat, elafin, secretory leukocyte protease inhibitor (SLPI), Prolastin and Aralast.

Modified derivatives (variants) of the peptides of the invention include those which vary from the particular sequence represented in SEQ ID NO: 1 by at least one amino acid, but which still possesses biological activity, i.e. the ability to inhibit the proteolysis of IGFBP-3 by at least about 20, 30. 40, 50, 60, 70, 80, 90, or 100% (or even more, i.e. the variant may be a better inhibitor), compared to the LIRCAML peptide. For example, one or more amino acids (e.g. 1, 2, 3, 4, 5, 6, or 7 amino acids) in the sequence may be substituted by another natural or non-natural amino acids, e.g. by Ala, Arg, Asn, Asp, Cys, Glu, Gin, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, a-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). In certain embodiments, the peptides are modified by C-terminal amidation, head to tail cyclic peptides, or containing Cys residues for disulfide cyclization, siderophore modification, or N-terminal acetylation.

Generally, variant peptides will display at least about 50, 55, 60, 65, 70, 75, 80, 85, 90, 95, 96, 97, 98, or 99% identity (homology) with the sequence of represented in SEQ ID NO: 1 (LIRCAML). For this peptide, for example, one or more of the hydrophobic amino acids L and I (and in some aspects, A) may be replaced by other hydrophobic residues (e.g. L, I, V, A, P, W, etc.), and the positively charged residue R may be replaced by the positively charged residue R, etc.

“Variant” peptides also include peptides derived from the native peptide by deletion (so-called truncation) or addition of one or more amino acids to the N-terminal and/or C-terminal end of the native peptide; deletion or addition of one or more amino acids at one or more sites in the native peptide; or substitution of one or more amino acids at one or more sites in the native peptide. The peptides of the invention may be altered in various ways including amino acid substitutions, deletions, truncations, and insertions. Methods for such manipulations are generally known in the art. Such peptide derivatives can be prepared, for example, as disclosed in U.S. Pat. Nos. 4,612,302; 4,853,371; and 4,684,620. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gin; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains.

The invention encompasses isolated or substantially purified peptide compositions. In the context of the present invention, an “isolated” or “purified” peptide is a peptide that exists apart from its native environment and is therefore not a product of nature. An isolated peptide may exist in a purified form or may exist in a non-native environment such as, for example, a transgenic host cell or bacteriophage. For example, an “isolated” or “purified” peptide, or biologically active portion thereof, is substantially free of other cellular material, or culture medium when produced by recombinant techniques, or substantially free of chemical precursors or other chemicals when chemically synthesized.

Various experimental techniques may yield peptides that are biologically active but have unfavorable pharmacological properties, such as difficulty to produce in large quantities, and sensitivity to protease digestion. Because peptides may be poor drug candidates, a need may arise for bioequivalent mimetic compounds which overcome these problems. These peptide mimics are inexpensive nonpeptidic oligomers and polymers that adopt amphiphilic secondary structures and exhibit potent and selective targeted activity similar to that of the peptide. Starting from a known spatial structure of a peptide template, the aim is to find compounds that mimic the function of a peptide but have e g improved cellular transport properties, low toxicity, few side effects and more rigid structures as well as protease resistance. Peptide mimetics may have several potential advantages over native peptides, such as increased stability, increased lipophilicity, increased rigidity, decreased size, and affordability of production.

Various methods exist for developing peptide mimetics. These include computational as well as experimental screening methods. Once a peptide of interest is identified, mimetics for the peptide are designed that can be used as drugs. On the basis of a known peptide sequence and/or structure, scaffolding templates can be constructed and then optimized using computerized methods. Peptide mimetics for the peptides disclosed herein encompass, for example, amphiphilic cationic molecules, e.g., substituted acrylamides. Candidate molecules may be screened using high-throughput screening techniques.

In certain aspects, the peptides of the present invention may be produced as fusion protein of the peptide sequence and a carrier protein. The carrier protein can subsequently be cleaved in vivo to release the active peptide in vivo. Phage display of active peptides may also be a useful method to present the peptide to cells for treatment of a subject.

The present invention provides compositions which comprise the protease inhibitors described herein. The compositions include one or more substantially purified inhibitory agents, and a pharmacologically suitable carrier. The preparation of such compositions is known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active agent in the formulations may vary. However, in general, the amount in the formulations will be from about 1-99%. Still other suitable formulations for use in the present invention can be found, for example in Remington's Pharmaceutical Sciences, Philadelphia, Pa., 19th ed. (1995).

The peptide compositions (preparations) of the present invention may be administered by any of the many suitable means which are well known to those of skill in the art, including but not limited to by injection, inhalation, orally, intravaginally, intranasally, by ingestion of a food or probiotic product containing the peptide, topically, as eye drops, via sprays, etc. In some aspects, the mode of administration is topical or orally or by injection. In addition, the compositions may be administered in conjunction with other treatment modalities such as substances that boost the immune system, various chemotherapeutic agents, anti-obesity agents, anti-DM2 agents, and the like.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

As indicated above, the present invention inter alfa provides the specified compound for use in a method of preventing or treating a chronic inflammation (obesity)-related disorder. For the avoidance of doubt, in this aspect the present invention may provide the specified compound for use as a medicament in the specified method. Further, the present invention may provide the specified compound as an active therapeutic ingredient in the specified method. Further, the present invention may provide the specified compound for use in a method of treatment of the human or animal body by therapy, the method comprising the specified method.

With regard to a peptide, a peptide consists essentially of a specified amino acid sequence if it does not include any additional amino acid residues at its amino and/or carboxyl terminus (except as described above for variants of the peptides), or if does not include any additional amino acid residues that occur adjacent to the peptides sequence in nature, i.e. in a natural product. In other words, a peptide sequence of the invention may be embedded in a sequence or attached to a sequence or, when located within a larger peptide or polypeptide, be flanked by sequences, with which it is not associated in nature. In addition, the peptide may include additional non-peptide components, such as labels (for example, fluorescent, radioactive, or solid particle labels), sugars, lipids, etc.

The term “treating” as used herein may refer to preventing the condition or disorder, slowing the onset or rate of development of the condition or disorder, reducing the risk of developing the condition or disorder, preventing or delaying the development of at least one symptom associated with the condition or disorder, reducing or ending at least one symptom associated with the condition or disorder, generating a complete or partial regression of the condition or disorder, or some combination thereof.

The dose of protease inhibitor that is administered is generally in the range of from about 1 nM to about 1000 nM, and more usually is in the range of from about 200 nM to about 500 nM. In cases where IGFBP-3 protein is administered, the dose is generally in the range of from about 1 nM to about 500 nM, and more usually is in the range of from about 50 nM to about 250 nM.

Diseases/conditions related to chronic inflammation (obesity) that may be prevented or treated as described herein include but are not limited to: chronic inflammation (obesity)-induced Type 2 DM, severe corticosteroid-dependent asthma, obstructive pulmonary disease, cardiovascular disease, cancer, metabolic syndrome and Th-1 Type inflammatory diseases, including but not limited to rheumatoid arthritis, juvenile arthritis, Crohn's disease, psoriasis, sarcoidosis, and Behcet's disease.

As used herein, cardiovascular disease (also called heart disease) refers to a class of diseases that involve the heart, the blood vessels (arteries, capillaries, and veins) or both. This includes any disease that affects the cardiovascular system, principally cardiac disease, vascular diseases of the brain and kidney, and peripheral arterial disease. These include but are not limited to: cardiomyopathy, hypertensive heart disease, heart failure, inflammatory heart disease, endocarditis, inflammatory cardiomegaly, myocarditis, valvular heart disease, cerebrovascular disease, peripheral arterial disease, etc.

A “metabolic syndrome” refers to one or more risk factors or symptoms commonly associated with overweight and obese subjects, which increases the risk to the subject of heart disease, diabetes, stroke, and other diseases associated with biochemical processes of the body. For example, metabolic syndrome may comprise one or more symptoms, including, but not limited to: insulin resistance, hyperlipidemias, hypertension, atherosclerosis, any chronic inflammation (obesity)-induced metabolic dysregulation, and diseases attributed to elevated NF-κB activity (e.g., inflammatory disease, Duchenne muscular dystrophy), among others. Although subjects having metabolic syndrome are often obese and overweight, a non-obese or non-overweight subject exhibiting one or more of the above symptoms can be a candidate for the methods and compositions disclosed herein.

Cancers (carcinomas) that may be detected or for which a predisposition may be detected, or treated include: cancers derived from epithelial cells, e.g. breast, prostate, ovaries, lung, pancreas, liver, colon, rectal, etc.; sarcomas; lymphoma and leukemia, germ cell tumor; blastomas; etc.

### Apparatuses and Computer-Executable Programs

The invention also provides apparatuses which carry out or aid in carrying out the diagnostic and monitoring methods of the invention. The apparatuses typically comprise: a detection system for detecting one or more of the biomarkers described herein in a biological sample; a first unit for processing input from the detection system; and a second unit for displaying an amount of the biomarkers that is detected. In some embodiments, the detection system comprises one or more of a high performance liquid chromatography apparatus, a liquid chromatography-mass spectrometer, a gas chromatography-mass spectrometer, an enzyme reagent reaction apparatus, a chemical reagent reaction apparatus, an electrophoresis apparatus, a nuclear magnetic resonance apparatus, an ultracentrifugation apparatus, a spectrometer using an ultraviolet ray, and a potential difference measuring apparatus. In other embodiments, the apparatus also includes a third unit that classifies the biological sample as corresponding to a healthy subject or a patient with chronic inflammation (obesity)-related disorder based on the amount of biomarker. The apparatus may also include a fourth unit that determines the degree of progression of a detected chronic inflammation (obesity)-related disorder based on the amount of biomarker.

Other apparatuses are also contemplated. For example, apparatuses which include i) a measuring system that measures the concentration of chronic inflammation (obesity)-related contained in a biological sample collected from a subject; ii) an input system that inputs the chronic inflammation (obesity)-related concentration measured by the measuring system; and iii) a computing system that determines at least one of the presence of an chronic inflammation (obesity)-related disorder, severity of the disorder, and a therapeutic effect on the disorder based on the biomarker concentration inputted from the input system. Suitable measuring systems include an element (unit) such as those described above, e.g. a detection system for detecting/determining a concentration of biomarker in a biological sample; a first unit for processing input from the detection system (e.g. for processing the detected concentration of biomarker, for comparing the amount to references values, for generating output in suitable units, etc;) and a second unit for displaying an amount of biomarker in a suitable format, e.g. on a computer screen, on the display of a handheld device, as a printed hard copy, etc.

The invention also provides computer-executable programs stored on non-transient media for carrying out the methods described herein. In one embodiment, the computer executable program is for diagnosing chronic inflammation (obesity)-related disorders, and includes instructions which cause a computer, or a plurality of networked computers, to execute at least the following steps: a step in which input means inputs the concentration of biomarker contained in a biological sample collected from a subject into a computer having the input means and computing means; and a step in which the computing means determines one or more of: the presence of an chronic inflammation (obesity)-related disorder, severity of the disorder, and a therapeutic effect on the disorder based on the biomarker concentration inputted in the inputting step above. In some embodiments, the biological sample is a blood or serum sample, and the input means inputs the concentration of biomarker in the sample. The input may be generated and output from, for example, a high performance liquid chromatography apparatus, a liquid chromatography-mass spectrometer, a gas chromatography-mass spectrometer, an enzyme reagent reaction apparatus, a chemical reagent reaction apparatus, an electrophoresis apparatus, a nuclear magnetic resonance apparatus, an ultracentrifugation apparatus, a spectrometer using an ultraviolet ray, or a potential difference measuring apparatus, or a biomarker concentration outputted from a recording apparatus which records a biomarker concentration measured by the recording apparatus. The computing means then classifies a subject as a healthy subject or a patient with a chronic inflammation (obesity)-related disorder based on the biomarker concentration that is inputted for the subject. In some aspects, the computing means also determines the degree of progression of a disorder in the subject based on the biomarker concentration, and classifies the patient accordingly. For example, reduced blood intact IGFBP-3 concentration (<4500 ng/ml) along with presence IGFBP-3 fragments (28-, 27-, 25-20-18-kDa fragments) in adult individuals may be indicative of progression to insulin resistance and further development of T2DM, cardiovascular disease and other metabolic disorders. The computing means may also have the capacity to classify a subject identified as a patient with a chronic inflammation (obesity)-related disorder for the degree of progression the disorder based on the concentration of biomarker. For example, the degree of progression may be defined by Normal (intact IGFBP-3>4500 ng/ml with minimal fragments), Mild IR/prediabetes (intact IGFBP-3>4100<4500 ng/ml with significant IGFBP-3 fragments) and Severe IR/T2DM (intact IGFBP-3<4100 ng/ml with significant IGFBP-3 fragments). The degree of progression may be also defined by Normal (ratio of IGFBP-3 fragments of total IGFBP-3 equal to or smaller than the first pre-determined reference level); Mild IR/prediabetes (ratio of IGFBP-3 fragments greater than 25% but less than 50% compared to the first pre-determined reference level) and Severe IR/T2DM (ratio of IGFBP-3 fragments greater than 50% compared to the first pre-determined reference level).

Thus, the computing means may classify the subject as a healthy subject or a patient with a chronic inflammation (obesity)-related disease, or at risk of developing an chronic inflammation (obesity)-related disease, or in the early stages of such a disorder, based on the biomarker concentration detected in the sample from the patient.

### Kit

The invention also provides kits for use in measuring the level of biomarker (e.g. proteolytic fragment(s) and/or IGFBP-3 levels and/or neutrophil protease levels) in a biological sample. The kits generally comprise one or more standard solutions of known concentrations of biomarker that are used to establish a standard curve, or solid or particulate biomarker that can be formulated into standard solutions. As is known in the art, standard curves are generally determined by measuring a plurality of quantities of a compound of interest using a technique of interest, e.g. chromatography, mass spectroscopy, etc. Standard concentrations are formulated to deliver, to a measuring device, a plurality of quantities of biomarker that bracket the likely concentrations of biomarker in an experimental sample, e.g. in a sample with an unknown concentration of biomarker. In other words, the plurality of quantities generally include some that are greater than and some that are less than the likely amount of biomarker in the sample. For example, in order to establish the concentration of biomarker in a biological sample, control standards are provided at concentrations which allow the delivery of known amounts of biomarker. The known quantities of biomarker are delivered to and measured or detected using the system or device that is employed to carry out the analysis.

In some aspects, the kit comprises a single sample (bottle, ampoule, or other container) of biomarker in solution at a concentration that can be diluted as necessary or desired by the end-user of the kit in order to provide multiple standard solutions of varying concentrations of the biomarker. Alternatively, the kit may include multiple standard solutions, each with a known concentration of biomarker, or the kit may include solid biomarker in a dried or crystalline form, e.g. as a lyophilized powder, for reconstitution by the end-user.

### General Caveats

The concentrations of the standard solution(s) are formulated so as to be sufficient to deliver the requisite or desired amounts of the biomarker of interest in a reasonable or suitable volume. Exemplary standard concentrations range from e.g. about 1×10⁻⁵ to about 1×10⁻¹⁰ moles/liter, and usually from about 1×10⁻⁶ moles/liter to about 1×10⁻⁹ moles per liter. A suitable volume may be, for example, from about 0.5 μl to about 1000 μl, e.g. from about 1 to about 500 μl, or about 1, 10, 20, 30, 40, 50, 60, 70, 80, 90, 100, 200, 300, 400 or 500 μl of solution, depending on the technique or device that is used to perform the measurements. Any suitable solvent may be used to form the biomarker standard solutions, so long as the biomarker is dissolved or dispersed therein and is stable in the solvent (at least for a period of time sufficient to conduct an assay). Exemplary solvents that may be employed include but are not limited to aqueous based solvents or buffering solutions (e.g. saline, phosphate, acetate, etc.) which can fully solubilize the biomarker. The biomarker that is present in the kit is/are typically chemically synthesized and substantially purified.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Before exemplary embodiments of the present invention are described in greater detail in the Examples below, it is to be understood that this invention is not limited to particular embodiments described, as such may, of course, vary. It is also to be understood that the terminology used herein is for the purpose of describing particular aspects only, and is not intended to be limiting, since the scope of the present invention will be limited only by the Examples or appended claims.

### EXAMPLES

### Example 1

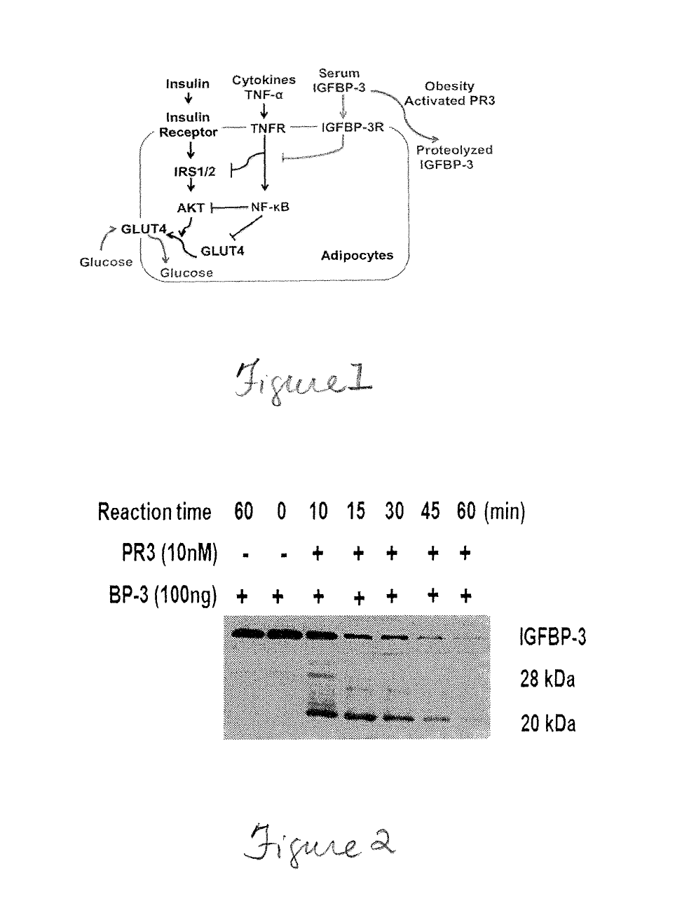

Visceral fat obesity correlates significantly with insulin resistance, hypertension and cardiac dysfunction. Chronic low-grade adipose tissue and liver inflammation is a major cause of systemic insulin resistance and is a key component of the low degree of insulin sensitivity that exists in obesity and T2DM. In particular, in the obese state, visceral adipocytes secrete pro-inflammatory cytokines such as TNF-α, CRP and IL-6 and produce less adiponectin, an adipocyte-derived hormone with anti-inflammatory and insulin-sensitizing properties. These metabolic changes likely contribute to lowered insulin sensitivity. PR3, a neutrophil serine protease, is secreted from the activated neutrophils and is critically involved in bacterial defense, but also regulates non-infectious inflammatory processes by modulating the activities of cytokines such as TNF-α, IL-1β, IL-8, IL-18 and IL-32. Recent studies further suggest that PR3 as well as NE and CG, might contribute to neutrophil-dependent inflammation and progression of chronic inflammatory diseases including diabetes, cystic fibrosis and glomerulonephritis8. The IGF system plays an important role in growth, development and maintenance of homeostasis in normal cells9. IGFBP-3, the major binding protein in circulation has been shown to be associated with obesity, insulin resistance, diabetes and cardiovascular diseases. These findings strongly suggest that IGFBP-3/IGFBP-3R system plays an important regulatory role in chronic inflammation (obesity)-induced insulin resistance, and that reduced levels of IGFBP-3 in circulation in chronic inflammation (obesity) may result in suppression of its anti-inflammatory, insulin sensitizing functions (FIG. 1).

Data presented in this Example supports the hypothesis that weight-associated increases in the activity of neutrophil serine proteases results in reductions of serum IGFBP-3 and abrogates an anti-inflammatory and insulin-sensitizing IGFBP-3/IGFBP-3R cascade in fat, muscle and liver. These important metabolic changes contribute to the development of systemic insulin resistance during the transition from being lean to being overweight to being obese.

### PR3 is a Specific Protease for IGFBP-3

Since it has been reported that PR3 appears to contribute to progression of chronic inflammatory diseases, we characterized PR3 as a specific IGFBP-3 protease in obese condition. The experiments were carried out using recombinant PR3 and IGFBP-3, and Western immunoblotting with IGFBP-3 specific antibody.

The results are shown in FIG. 2. As can be seen, recombinant IGFBP-3 was proteolyzed by PR3 in a time dependent manner. Significantly, treatment with PR3 at physiological concentration (10 nM) resulted in a significant IGFBP-3 proteolysis during a 10 min incubation at 37° C. These data show that PR3 is capable of proteolyzing IGFBP-3 under physiological conditions.

### PR3 and IGFBP-3 Proteolysis in Insulin Resistant Obese Population

We next ascertained the levels of PR3 and IGFBP-3 proteolysis in a population of insulin resistant obese subjects. The experiments were carried out using serum samples from lean, overweight and obese adolescence.

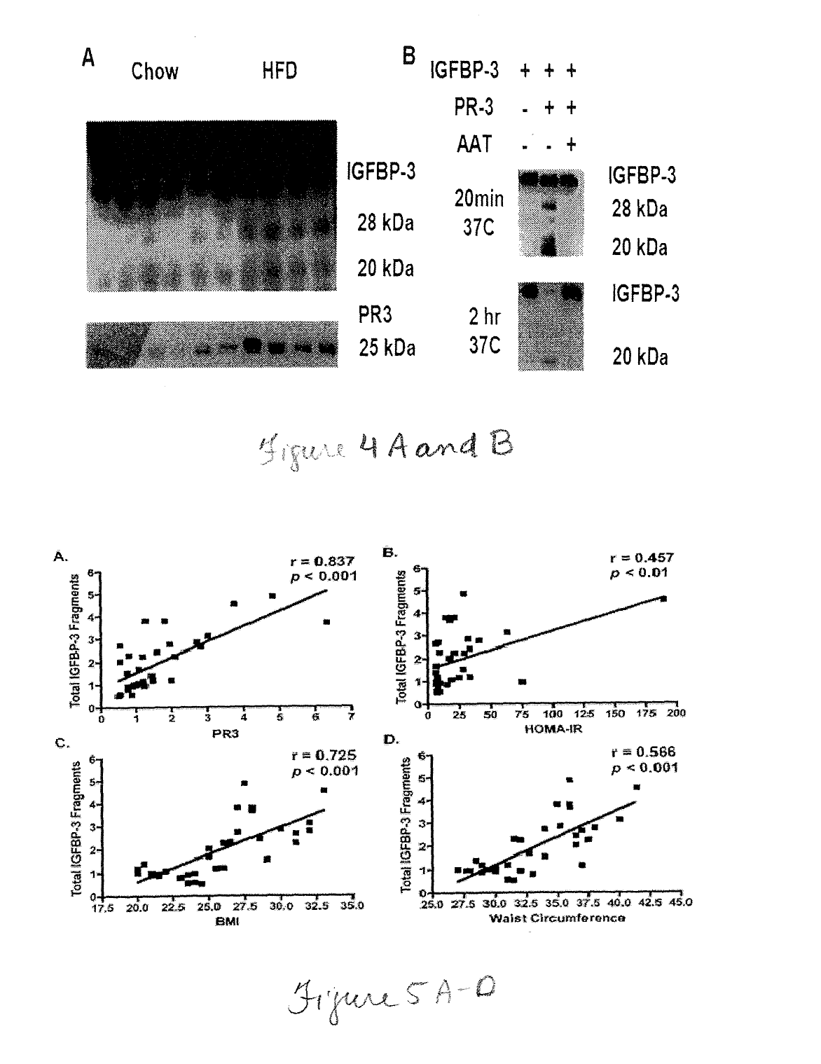

The data, which is presented in FIG. 3, showed a significant increase in levels of PR3 and in IGFBP-3 proteolysis in these subjects. In particular, IGFBP-3 proteolyzed fragments and PR3 are increased in insulin resistant obese population. A positive correlation exists between IGFBP-3 proteolysis and PR3 (A), whereas an inverse correlation exists between whole body insulin sensitivity index and IGFBP-3 proteolysis (B); as well as PR3 levels (C). n=9. D, representative Western immunoblot analysis of IGFBP-3 proteolysis and PR3 levels in insulin-sensitive (high whole-body insulin sensitivity index, “WBISI”) and insulin-resistant population (WBISI<2.0).

Taken together, this data shows that systemic inflammation in obesity is associated with increased levels of PR3 in serum and with a significant increase of serum IGFBP-3 proteolysis.

### Example 2

Insulin resistance (IR) represents a common metabolic derangement that contributes to the development of many chronic inflammation (obesity)-related comorbidities including type 2 diabetes mellitus (T2DM). Although it is generally established that low-grade adipose tissue inflammation contributes to the burden of IR, the pathophysiology underlying the development of IR is complex. In addition to alterations in other metabolic pathways, perturbations in the growth hormone/insulin-like growth factor-1 (IGF-1) axis have been implicated in the process. Levels of a specific IGF-1 binding protein, IGFBP-3, are associated with chronic inflammation (obesity), IR and diabetes. It has been previously demonstrated that, through activation of a dedicated receptor (IGFBP-3R), IGFBP-3 inhibits cytokine-induced NF-κB activity and improves insulin signaling in human adipocytes. Furthermore, it has been shown that obese adolescents demonstrate reductions in total IGFBP-3 and increases in proteolytic fragments of IGFBP-3 when compared with their non-obese counterparts IGF-BP3. However, the mechanisms responsible for IGFBP-3 degradation are unclear.

Neutrophil serine proteases (NSPs) are released at sites of inflammation and activate pro-inflammatory cytokines. Although classically associated with innate immunity and pathogen destruction, NSPs are also involved in the pathogenesis of many chronic inflammatory conditions and are activated in the setting of obesity, In addition to directly secreting pro-inflammatory cytokines, adipocytes further enhance the inflammatory milieu in obesity by recruiting in situ additional inflammatory cells including macrophages and lymphocytes. Proteinase 3 (PR3) is secreted from activated neutrophils and recent studies suggest that PR3, as well as other neutrophil serine proteases (NSPs) such as neutrophil elastase (NE) and cathepsin G (CG), contribute to neutrophil-dependent inflammation and progression of chronic inflammatory diseases including diabetes, cystic fibrosis and glomerulonephritis. Conversely, NSP inhibitors such as α-1-antitrypsin (AAT) have been proposed as treatments in patients with chronic inflammatory diseases.

The experiments described in this Example investigated the relationship between chronic inflammation (obesity)-induced activation of inflammatory NSPs and reductions in levels of intact, biologically-active IGFBP-3, as well as contributions thereof to development of systemic IR. This research led to the discovery of interventions which prevent and/or treat chronic inflammation (obesity)-induced inflammation and diseases and conditions associated therewith, e.g., by reducing NSP-induced IGFBP-3 proteolysis.

Since PR3 appears to contribute to progression of chronic inflammatory diseases, the role of PR3 as a specific IGFBP-3 protease in obese conditions was characterized. As shown in FIG. 4A, serum PR3 and IGFBP-3 proteolytic fragments were significantly increased in HFD mice compared to those on a chow diet (CD). Furthermore, recombinant IGFBP-3 was proteolyzed by PR3 while AAT completely inhibited PR3-induced IGFBP-3 proteolysis (FIG. 4B).

In order to further clarify the role of PR3 in chronic inflammation (obesity) induced IGFBP-3 proteolysis in human and its correlation with obesity, 34 serum samples of lean (n=14), overweight (n=14) and obese (n=6) women were analyzed. The results are presented in FIG. 5A-D. As can be seen, an increase in proteolyzed IGFBP-3 as well as PR3 was observed in circulation in overweight and obese populations when compared to lean counterparts. Furthermore, IGFBP-3 proteolysis is positively correlated with PR3 levels and waist circumference, body mass index (BMI) and insulin resistance (HOMA-IR). Furthermore, predisposition to IR and IGFBP-3 proteolysis appears to occur not only in obese populations but also in overweight individuals, strongly suggesting that IGFBP-3 proteolysis is a biomarker for a predisposition to insulin resistance.

Similarly, the increased levels of all NSPs, PR3, NE and CG as well as IGFBP-3 fragments in the circulation were observed in overweight (BMI 25-29.9) and obese (BMI>30) human individuals compared to lean counterparts (BMI 18.5-24.9) (FIG. 6A-D). In human serum, proteolyzed IGFBP-3 levels are increased (bands at 28 and 18 kDa) in the circulation of overweight (n=3) and obese (n=3) individuals when compared with their non-obese counterparts (n=3). and their appearance gradually increases during progression to overweight and then obese conditions. These findings suggest that increased IGFBP-3 proteolysis in overweight and obese individuals likely results in reduced levels of functional IGFBP-3 in the circulation, effectively blunting the anti-inflammatory and insulin-sensitizing functions of IGBPB-3 in adipose tissue. Furthermore, evidence of increased IGFBP-3 proteolysis is already present even in overweight individuals, suggesting a potential role of IGFBP-3 proteolysis as a biomarker for predisposition to IR. Interestingly, among all 3 NSPs, PR3 and NE are significantly increased in overweight and obese condition whereas CG appears to be increased only in the obese condition.

Further, in vitro proteolysis experiments demonstrated that all three of these NSPs proteolyze IGFBP-3. As shown in FIG. 6B, treatment with PR3 at a concentration of 100 nM resulted in a significant reduction of intact IGFBP-3 while increasing 28 kDa and 20 kDa fragments. Higher concentration of the protease further decreased intact IGFBP-3 and increased the level of fragments.

Taken together, this data shows that systemic inflammation in obesity increases NSP activities, which causes a significant increase of IGFBP-3 proteolysis. This would likely abrogate the endocrine/paracrine/autocrine derived insulin-sensitizing actions of IGFBP-3 in insulin target tissue including visceral fat, thereby resulting in IR.

### Example 3

### Determination of Threshold Values of Intact IGFPB-3 and IGFBP-3 Fragments

It is of great interest to determine thresholds of intact and proteolyzed IGFBP-3 during progression of disease. Experiments were conducted using two different methods to identify status of intact IGFBP-3 vs IGFBP-3 fragment in blood samples: intact IGFBP-3 specific assay and Western immunoblotting for quantification of IGFBP-3 fragments. As shown in FIG. 7A-D, the amount of intact-IGFBP-3 measured by ELISA in lean group (4600±128.2 ng/ml) was significantly higher than that in overweight (4166±150.8 ng/ml) and obese groups (3909±176.8 ng/ml). p<0.05 (A). There is a significant correlation between the amount of intact IGFBP-3 and the ratio of intact IGFBP-3 and total IGFBP-3 (r=0.4776, p<0.05) (B). IGFBP-3 proteolysis in lean group (0.44±0.03) was significantly low than that in overweight (0.60±0.04) and obese (0.69±0.04) groups. p<0.01 (C). There is an inverse correlation between the amount of intact IGFBP-3 and IGFBP-3 proteolysis (r=0.4776, p<0.05) (D).

### Example 4

### NP Inhibition In Vitro

It is of great interest to identify inhibitors of NPs in order to intervene in the processes that lead to diseases caused by chronic inflammation (obesity). For example, it is of great interest to generate and characterize recombinant peptide inhibitors of NSPs that can be readily and inexpensively synthesized. Due to its molecular and structural complexity (394 amino acids with a number of disulfide linkages and glycoforms), current clinical formulations of NSP inhibitors are natural forms of AAT which are purified from pooled human plasma, a costly and time-consuming method.

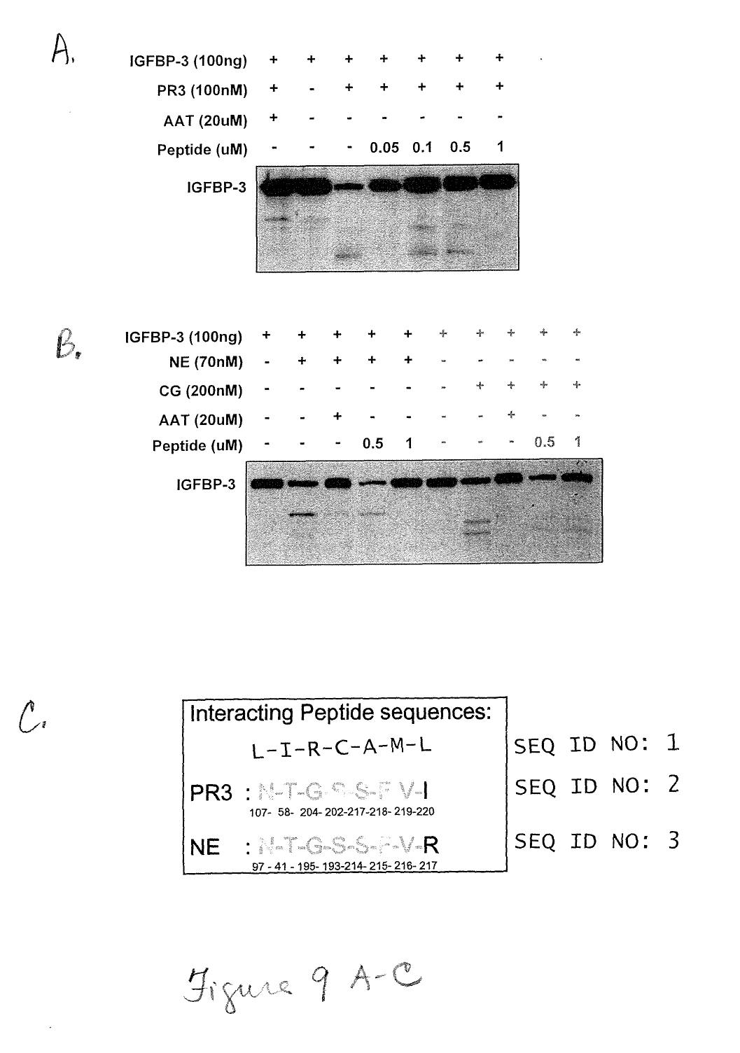

Experiments were conducted to identify the functional domains of NSPs, especially the domains that interact directly with (e.g. bind to) AAT. Based upon the sequence identity among NSPs PR3, NE and CG at a putative binding site of AAT, a 7 amino acid-long peptide was generated and tested for its ability to block NSP binding to substrates (e.g. IGFBP3) and thus prevent or attenuate NSP protease activity. As shown in FIGS. 8 and 9A-C, the small peptide inhibitor (LIRCAML, SEQ ID NO: 1) displays a potent inhibitory effect on PR-3-induced IGFBP-3 proteolysis at concentrations ranging from 0.5 μM to 20 μM in a dose-dependent manner. Even treatment with the small peptide inhibitor at a concentration of 0.5 μM resulted in more than 50% inhibition of IGFBP-3 proteolysis induced by 100 nM PR3 treatment. The inhibitory effect of the small peptide inhibitor on PR3 activity at the concentration of 20 μM was comparable to that of 100 nM AAT. These data show that the small peptide inhibitor (LIRCAML, SEQ ID NO: 1) is a potent NSP inhibitor, blocking NSP-induced IGFBP-3 proteolysis. This peptide and/or suitable variants thereof can thus be used to treat individuals with chronic inflammation (obesity)-induced insulin resistance.

These findings suggest that chronic inflammation (obesity)-induced activation of NSPs leads to proteolysis of IGFBP-3 in circulation and in tissue, abrogating the anti-inflammatory, insulin-sensitizing IGFBP-3/IGFBP-3R cascade in circulation, and in fat, muscle and liver during the transition from lean to overweight to obese conditions, resulting in insulin resistance. Therefore, restoration of a functional IGFBP-3/IGFBP-3R cascade (e.g. by administration of an agent that could prevent or reverse this abrogation) would prevent/reverse chronic inflammation (obesity)-induced insulin resistance.

### Example 5

### Anti-Inflammatory and Insulin Sensitizing Effects of the Small Peptide Inhibitor (LIRCAML, SEQ ID NO: 1)

Since PR3, NE and CG appear to be involved in inflammation and metabolic disorders, all three proteases are characterized with respect to IGFBP-3 proteolysis in vitro. The in vitro IGFBP-3 protease assay is initially performed using recombinant IGFBP-3, NSPs (PR3/NE/CG) and their inhibitors AAT and the small peptide inhibitor (LIRCAML, SEQ ID NO: 1) in order to determine the specificity and the optimal concentration of each protease and inhibitor on IGFBP-3 proteolysis.

The results show that increased PR3 (or NE and CG) activity increases IGFBP-3 proteolysis whereas NSP-induced proteolysis of IGFBP-3 is blocked by AAT and the small peptide inhibitor(s).

### Example 6

### NSP Inhibitor Administration Prevents/Reverses IR in HFD-Induced Obese Mice

It is of great interest to identify diagnostic and therapeutic potential of NSP inhibitors and IGFBP-3 for chronic inflammation (obesity)-induced insulin resistance. Therefore, 1) serum PR3, NE, CG, IGFBP-3 and IGFBP-3 proteolysis were tested as potential indicators for chronic inflammation (obesity)-induced IR; and 2) administration of PR3 inhibitors (a clinical formulation of AAT, Aralast and small peptide inhibitor(s)) will prevent/reverse IR in a HFD-induced obese mouse model.

The effect of AAT, Alatast on obesity-induced insulin resistance was tested using C57BL6/J BomTac mice feeding with CD and HFD for 15 weeks (n=3 mice per group). Assessment of body weight, fed and fasting glucose, intraperitoneal glucose tolerance test (IPGTT) and insulin tolerance test (ITT) were performed at 8, 15 weeks of HFD.

In order to assess the potential of IGFBP-3 and NSP inhibitors as early interventions for chronic inflammation (obesity)-induced IR, two mouse models were employed (n=3 mice per group): 1) HFD mice and 2) HFD mice with AAT (Aralast) administration. Mice were fed with a HFD for 15 weeks; and the following were preformed: 1) intraperitoneal treatment with PBS or 2) intraperitoneal treatment with clinical formulations of AAT, Aralast at a dose of 60 mg/kg body weight once weekly, starting at 8 weeks of HFD feeding The dosage of Aralast (60 mg/kg body weight) was adopted from previous in vivo studies. Weekly assessment of body weight was performed. IPGTT and ITT were analyzed at 8, 15 weeks of HFD.

### Laboratory Methods:

Description of murine models: Six week old male C57BL6/J BomTac mice were obtained from Taconic, Hudson, N.Y., and kept at 22° C. on a 12:12-h light-dark cycle, with food and water ad libitum. All mice were placed on a normal rodent diet (ND) D12450B for 1 week and then divided into two groups and fed with ND and high fat diet (HFD) D12492 for 16 weeks. ND (D12450B, calories provided by fat 10%, protein 20%, Carbohydrate 70%) and HFD (D12492, calories provided by fat 60%, protein 20%, Carbohydrate 20%) were from Research Diets, new Brunswick, NJ52.

Intraperitoneal glucose tolerance test (IPGTT): After an 6 h fast, an intra-peritoneal bolus of glucose (2 mg/g body weight) was administered and blood glucose levels were determined by commercially available glucometer (AccuChek Plus) using tail vein blood at 0, 15, 30, 60, 120 and 180 minutes. Blood insulin levels were measured by Ultrasensitive mouse insulin ELISA kit (ALPCO Diagnostics).

Insulin tolerance test (ITT): After a 7-day recovery period, an ITT was performed on the same mice. After a 4 h morning fast (8 am to noon) and measurement of fasting blood sugar, mice were given a bolus (0.75 U/kg body weight) of insulin and blood glucose levels were measured at 15, 30, 60, 120 and 180 minutes.

Data Analysis: 1) Mice with HFD feeding and with/without NSP inhibitor treatments awere studied with assessment of body weight, fed and fasting glucose, IPGTT and ITT. Statistical analysis: Differences between groups are determined by ANOVA followed by Turkey post hoc tests or by Student t Test as appropriate. Significance is considered as p<0.05.

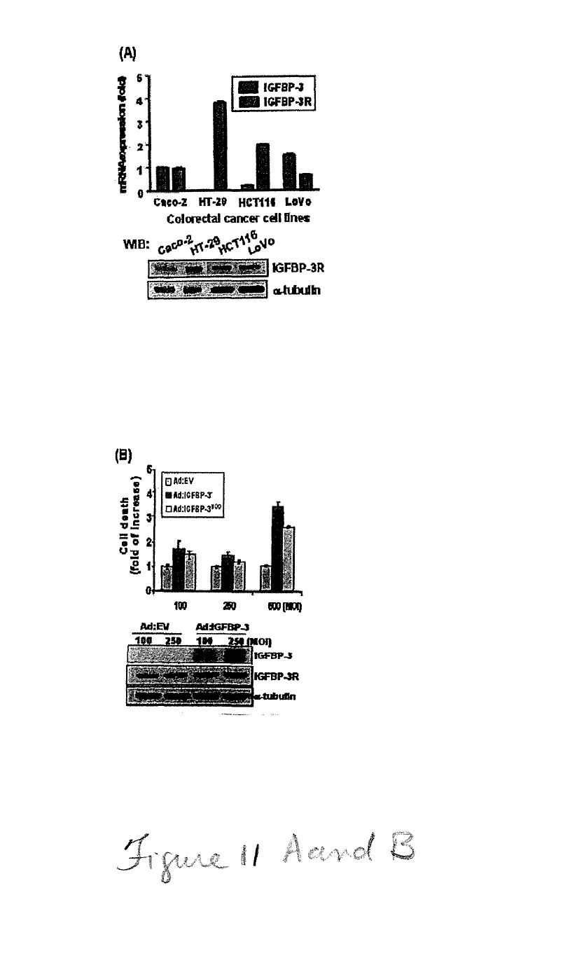

As shown in FIG. 10, mice have insulin resistance (IR) after 8 weeks of HFD feeding and treatment using the NSP inhibitor Aralast results in a subsequent increase of its insulin-sensitizing effects in the mice. Symptoms of insulin resistance are prevented in mice treated with Aralast from day 0 and reversed in mice treated with Aralrast later in the study.

### Example 7

The levels of the three NSPs and IGFBP-3 protease activity in sera from lean and obese mice fed with CD and HFD, respectively, for 16 weeks are investigated using Western immunoblotting, ELISAs and an IGFBP-3 protease activity assay. Further, the impact of PR3-induced proteolysis and inhibitors (Prolastin and a small peptide inhibitor) on insulin sensitizing effects of IGFBP-3 in primary adipocytes (Invitrogen/Life Technologies, Grand Islands N.Y.), myoblasts and hepatocytes (Eton Bioscience, Research Triangle Park, N.C.) is investigated. The cells are treated with IGFBP-3 or PR3 proteolyzed IGFBP-3 fragments with and without insulin, and analyzed for glucose uptake, levels of insulin receptor substrates (IRSs), glucose transporters (GLUTs), adiponectin (in adipocytes) and intracellular glycogen content (in hepatocytes) using a glucose uptake assay, qRT-PCR, Western immunoblotting and a glycogen assay kit. Further experiments are performed to treat cells with IGFBP-3 and PR3 in the presence and absence of Prolastin or the small peptide inhibitor(s) described herein. The inhibitory effects of NE and CG on IGFBP-3's insulin-sensitizing effects are also determined using similar laboratory methods. The involvement of IGFBP-3R on the insulin signaling pathway is also investigated by manipulating endogenous IGFBP-3R expression using IGFBP-3R siRNAs.

Laboratory Methods: Human and mouse specific IGFBP-3 antibodies (Abs), IGFBP-3R Abs, IGFBP-3 protease assay, and adenoviral plasmids transfused with IGFBP-3R or their small hairpin RNAs (shRNAs) are utilized in these investigations.

The results show that the proteases (NE, PR3 and CD) all produce significant IGFBP-3 proteolysis and inhibition of IGFBP-3's normal metabolic function. In addition, protease specific inhibitors of the proteases (e.g. Aralast, the small peptide inhibitor, GW311616A, etc.) reverse this effect.

### Example 8

### NSP Inhibitor Administration Prevents/Reverses IR in HFD-Induced Obese Mice

In order to assess the potential of IGFBP-3 and NSP inhibitors as early interventions for obesity-induced IR, three mouse models are employed (n=6 mice per group): 1) HFD mice, 2) HFD mice with AAT (Aralast) administration, and 3) HFD mice with small peptide inhibitor administration. Parallel studies are performed in which mice with CD are treated with NSP inhibitors. Mice are fed with a HFD or CD for 16 weeks; and the following are preformed: 1) intraperitoneal treatment with clinical formulations of AAT, Prolastin at a dose of 60 mg/kg body weight once weekly, starting at the first day (for preventive effects) (A) or at 8 weeks (for reverse effects) (B) of HFD feeding; and 2) mice are treated with the small peptide inhibitor (LIRCAML) (600 mg/kg body weight once weekly) starting at week 0 (C) or at week 8 (D). The dosage of Prolastin (60 mg/kg body weight) is adopted from previous in vivo studies, and the small peptide inhibitor (600 mg/kg body weight). Weekly assessment of body weight is performed. IPGTT and ITT are analyzed at 1, 8, 12, 16 weeks of HFD. In parallel, serum NPs, IGFBP-3 and proteolyzed IGFBP-3 are measured at 1, 5, 8, 12, 16 weeks using Western immunoblotting and ELISAs with mouse or human IGFBP-3 specific antibodies, and Western immunoblotting, and PR3 activity assays using InnoZyme PR3 immunocapture activity assay kit during HFD-induced obesity and progression to insulin resistance. The proteins/genes involved in insulin signaling and inflammatory signaling such as adiponectin, leptin, [[TNF-α,]] MCP-1, CRP, MCP-1, IRSs and GLUTs are also investigated in serum, as well as in total protein lysates and mRNAs of adipose tissue, muscle, and liver, using Multiplex immunoassays, Western immunoblotting and qRT-PCR, respectively. Hyperinsulinemic-euglycemic clamp studies are performed using the HFD mice treated with/without AAT for 16 weeks (n=6) at the Yale Mouse Metabolic Phenotyping Center, Yale University School of Medicine.

A separate experimental group of HFD mice are treated with a NE specific inhibitor, GW311616A, to determine significance of NE on IGFBP-3 proteolysis and HFD-induced IR39. Another separate experimental group of mice are further treated with both NSP inhibitor and IGFBP-3 (3 mg/kg body weight once weekly).

### Laboratory Methods:

Description of murine models: Six week old male C57BL6/J BomTac mice are obtained from Taconic, Hudson, N.Y., and kept at 22° C. on a 12:12-h light-dark cycle, with food and water ad libitum. All mice are placed on a normal rodent diet (ND) D12450B for 1 week and then divided into two groups and fed with ND and high fat diet (HFD) D12492 for 16 weeks. ND (D12450B, calories provided by fat 10%, protein 20%, Carbohydrate 70%) and HFD (D12492, calories provided by fat 60%, protein 20%, Carbohydrate 20%) are from Research Diets, new Brunswick, NJ52.

Intraperitoneal glucose tolerance test (IPGTT): After an 6 h fast, an intra-peritoneal bolus of glucose (2 mg/g body weight) is administered and blood glucose levels are determined by commercially available glucometer (AccuChek Plus) using tail vein blood at 0, 15, 30, 60, 120 and 180 minutes. Blood insulin levels re measured by Ultrasensitive mouse insulin ELISA kit (ALPCO Diagnostics).
Insulin tolerance test (ITT): After a 7-day recovery period, an ITT is performed on the same mice. After a 4 h morning fast (8 am to noon) and measurement of fasting blood sugar, mice are given a bolus (0.75 U/kg body weight) of insulin and blood glucose levels are measured at 15, 30, 60, 120 and 180 minutes.
Euglycemic-hyperinsulinemic clamp: The clamp studies are performed using the HFD mice treated with/without AAT for 16 weeks (n=6) at the Yale Mouse Metabolic Phenotyping Center, Yale University School of Medicine. Following an overnight fast, a 2 hr hyperinsulinemic-euglycemic clamp is conducted in conscious mice with a primed and continuous infusion of human insulin (150 mU/kg body weight priming followed by 2.5 mU/kg/min; Humulin; Eli Lilly), and 20% glucose is infused at variable rates to maintain euglycemia. Animals are anesthetized, and tissues are collected and rapidly frozen for subsequent analysis.
Data Analysis: 1) Mice with HFD or CD feeding and with/without NSP inhibitor treatments are studied with assessment of body weight, fed and fasting glucose, IPGTT and ITT. Insulin secretion and IR are assessed using the hyperglycemic and hyperinsulinemia clamp methods; 2) Mice serum IGFBP-3, proteolyzed IGFBP-3, and NSPs (PR3/NE/CG) are analyzed at 1, 5, 8, 12, 16 weeks of HFD; and 3) Insulin signaling and inflammatory signaling in adipose tissue, muscle, and liver are examined using qRT-PCR, immunohistochemistry, Western immunoblotting and fresh-frozen and paraffin-embedded tissue samples collected at week 0, 8, 12 and 16.
Statistical analysis: Differences between groups are determined by ANOVA followed by Turkey post hoc tests or by Student t Test as appropriate. Significance is considered as p<0.05. Association between IGFBP-3 proteolysis and PR3 (and/or NE, CG) or IR is assessed by a test of correlation.

The results show that mice have IR after 8 weeks of HFD feeding. Treatments using NSP inhibitors result in an increase of intact IGFBP-3 in circulation and a subsequent increase of its insulin-sensitizing effects in the mice. Symptoms of insulin resistance are prevented in mice treated from day 0 and reversed in mice treated later in the study.

### Example 9

### Relationship to Colon Cancer

The studies described above demonstrated that the NSPs NE, PR3 and CG specifically proteolyze IGFBP-3, and that increased IGFBP-3 proteolytic fragments in chronic inflammatory conditions are attributable to the increased activity of NSPs in circulation. Thus, since neutrophil activation is known to occur in the setting of colonic inflammation (colitis) and NSPs have been shown to be associated with IGFBP-3 proteolysis (see Examples above), it is likely that colitis-induced activation of NSPs leads to reductions in levels of intact, biologically-active IGFBP-3 in circulation as well as in colon tissue, and abrogating antitumor and anti-inflammatory functions of endocrine/paracrine/autocrine-derived IGFBP-3 during development of CAC. In addition, NSP inhibitors reduce colonic inflammation and CAC development via reduction of NSP-induced IGFBP-3 proteolysis.

We have investigated the existence of an IGFBP-3/IGFBP-3R system in colon cancer cells. The data clearly demonstrated heterogeneous expression of endogenous IGFBP-3 and IGFBP-3R in various colon cancer cells (FIG. 11A). When IGFBP-3 or IGFBP-3GGG (an IGFBP-3 mutant which only binds to IGFBP-3R but not IGFs) was overexpressed, a significant induction of apoptosis was observed (FIG. 11B), suggesting existence of a functional IGFBP-3/IGFBP-3R system in colon cancer cells.

### Example 10

The biological impact of NSPs (PR3, NE, and CG) on IGFBP-3 proteolysis and the antitumor actions of IGFBP-3/IGFBP-3R signaling is investigated, e.g. to characterize specific mechanisms by which NSPs modulate the biologic impact of IGFBP-3/IGFBP-3R on cell proliferation, apoptosis in CRC. Accordingly, the impact of NSPs on the antitumor and anti-inflammatory functions of IGFBP-3 in a variety of colon cancer cells (HT-29, Caco-2, HCT116 and LoVo) is investigated using recombinant IGFBP-3 (intact or NSP-proteolyzed fragments), NSPs and their inhibitors. The involvement of IGFBP-3R on IGFBP-3's effects is tested by manipulating endogenous IGFBP-3R expression using additional lentiviral-IGFBP-3-GFP, IGFBP-3R siRNAs, Adeno-IGFBP-3R, NSPs and protease inhibitors in the same cell systems.

### Materials:

Human and mouse specific IGFBP-3 antibodies (Abs)20, IGFBP-3R Abs20, IGFBP-3 protease assay, and adeno-, retro- and lenti-viral plasmids transfused with IGFBP-3, IGFBP-3R or their small hairpin RNAs (shRNAs) are utilized in these investigations.

### Data Analysis:

The differential impact of intact IGFBP-3 and IGFBP-3 fragments on induction of apoptosis, cell proliferation in CRC cells is quantified using a Cell death assay, WST assay and qRT-PCR and immunoblotting.

The results show that increased NSP activity increases IGFBP-3 proteolysis whereas NSP-induced proteolysis of IGFBP-3 is blocked by AAT and Aralast. Unlike treatment with intact IGFBP-3, treatment with NSP-induced IGFBP-3 fragments does not show antitumor and anti-inflammatory effects in CRC cells. NE specific inhibitor (GW311616A) is also tested.

Studies are also carried out to determine whether administration of the NSP inhibitor (Aralast) inhibits progression of CRC in a CAC mouse model.

First, serum NSPs, IGFBP-3 and IGFBP-3 proteolysis are determined during CRC development in AOM-DSS-treated mice with/without Aralast administration (60 mg/kg body weight once weekly) (6 mice/group). Serum NSPs (PR3,NE,CG), IGFBP-3, and proteolyzed IGFBP-3 and cytokines (TNF-β, IL-6, IL-1β) are analyzed biweekly using Western immunoblotting, IGFBP-3 protease activity assays and mouse specific ELISAs (ELISA kits for PR3 from USCN life science; NE from Biotang USA; CG from CusaBio; TNF-α from Thermo Scientific; and IGFBP-3 from Sigma).

The therapeutic potential of AAT (Aralast) as an intervention for CAC is assessed. AAT or placebo is administered to AOM-DSS-treated mice (60 mg/kg body weight once weekly) at a day before the first AOM injection (at 5 weeks of age) until the last treatment. After sacrificing, mice colons are removed and flushed with phosphate-buffered saline (PBS) for macroscopic inspection. Tumor counts are performed in a blinded fashion. Tumor volume (V) is calculated daily by measuring length (L) and width (W) of the tumor with calipers and using the following formula: V={W×L×[(W+L)/2]}×0.52. Using paraformaldehyde fixed or fresh frozen tissue the proteins/genes involved in cell proliferation and inflammatory signaling are examined using Ki67 staining, apoptosis detection kit (Roche Dignostics GmbH) as well as immunoblotting and qRT-PCR for TNF-α, IL-6, IL-1β, NF-κB, IκBκ, IGFBP-3 and IGFBP-3R.

Murine models: Four-week-old male mice (C57BL6/J are fed with chow diet (Lab Diet #5001; calories provides by fat 13.5%, protein 28.5%, carbohydrates 58.0%). Mice are acclimated for 1 week and then injected intraperitoneally (IP) with 12.5 mg/kg AOM. After 5 days, 2.5% DSS is given in the drinking water for 7 days, followed by 14 days of regular water. This cycle (21 days) is repeated twice and mice are sacrificed 10 days after the last cycle, at 16 weeks of age.
Statistical analysis: Differences between groups are determined by ANOVA followed by Turkey post hoc tests or by Student t Test as appropriate. Significance is considered as p<0.05. Association between IGFBP-3 proteolysis and NSPs, cytokines or tumor volume is assessed by a test of positive correlation.

### Results:

AOM-DSS-treated mice exhibit increased serum levels of inflammatory cytokines, NSPs and IGFBP-3 proteolysis. In addition, AOM-DSS mice develop tumors, whereas AAT administration decreases tumor incidence and size compared with untreated mice due to an increase of intact, functional IGFBP-3 in circulation and the tissue environment, and subsequent enhancement of antitumor and anti-inflammatory functions.

The results of the integrated in vitro and in vivo studies characterize the functional role of the NSP-IGFBP-3/IGFBP-3R system in CAC and allow initiation of Phase II clinical trial of Aralast to CAC patients. In addition, a combination treatment of Aralast and IGFBP-3 is undertaken, since there is no concern of drug-promoted cancers with IGFBP-3 due to its dual antitumor and anti-inflammatory properties compared with other immunosuppressive drugs such as corticosteroids, methotrexate and anti-TNF-α.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for early diagnosis of a subject having a tendency to develop chronic inflammation associated with obesity, comprising
contacting a biological sample from the subject with at least one agent which selectively binds to at least one biomarker of insulin-like growth factor-binding protein 3 (IGFBP-3) proteolysis by at least one neutrophil protease, wherein said step of contacting is carried out under conditions which allow the at least one agent to form an agent-biomarker complex with the at least one biomarker to which it selectively binds;
detecting a level of agent-biomarker complex in the sample;
comparing the level of agent-biomarker complex to at least one pre-determined reference level of agent-biomarker complex, wherein the at least one pre-determined reference level includes a first pre-determined reference level from a control population of individuals who do not have a tendency to develop chronic inflammation associated with obesity, and
i) if the level of complex differs from the first pre-determined reference level, then concluding that the subject has a tendency to develop chronic inflammation associated with obesity; and
ii) if the level of complex does not differ from the first pre-determined reference level, then concluding that the subject does not have a tendency to develop chronic inflammation associated with obesity.

2. The method of claim 1, wherein the at least one biomarker is selected from the group consisting of: one or more proteolytic fragments generated by cleavage of insulin-like growth factor-binding protein 3 (IGFBP-3) by a neutrophil protease; IGFBP-3; at least one neutrophil protease.

3. The method of claim 2, wherein said biomarker is IGFBP-3.

4. The method of claim 3, wherein i) levels of IGFBP-3 greater than 4500 ng/ml are considered normal; ii) levels of IGFBP-3 greater than 4100 ng/ml but less than 4500 ng/ml indicate early stage disease; and iii) levels of IGFBP-3 less than 4100 ng/ml indicate the presence of disease.

5. The method of claim 2, wherein the biomarker is at least one neutrophil protease.

6. The method of claim 5, wherein said at least one neutrophil protease is selected from the group consisting of proteinase 3 (PR3), neutrophil elastase (NE) and cathepsin G (CG).

7. The method of claim 2, wherein the biomarker is one or more proteolytic fragments.

8. The method of claim 7, wherein said one or more proteolytic fragments is selected from the group consisting of: a 28-kDa fragment formed by proteolysis of IGFBP-3 by PR-3, a 20-kDa fragment formed by proteolysis of IGFBP-3 by PR-3, a 25-kDa fragment by proteolysis of IGFBP-3 by NE, a 28-kDa fragment formed by proteolysis of IGFBP-3 by CG, a 27-kDa fragment formed by proteolysis of IGFBP-3 by CG, a 25-kDa fragment formed by proteolysis of IGFBP-3 by CG, a 20-kDa fragment formed by proteolysis of IGFBP-3 by CG, and a 18-kDa fragment formed by proteolysis of IGFBP-3 by CG.

9. The method of claim 1, wherein the at least one pre-determined reference level further includes a reference value selected from the group consisting of: a reference value from a control population of individuals who are developing an chronic inflammation (obesity)-associated disorder; a reference value from a control population of individuals who have an chronic inflammation (obesity)-associated disorder; and a reference value from the subject prior to receiving therapy to prevent an chronic inflammation (obesity)-associated disorder.

10. A method of diagnosing, in a subject in need thereof, whether or not the subject has or is developing an chronic inflammation (obesity) associated disorder, comprising
contacting a biological sample from the subject with at least one agent which selectively binds to at least one biomarker of insulin-like growth factor-binding protein 3 (IGFBP-3) proteolysis by at least one neutrophil protease, wherein said step of contacting is carried out under conditions which allow the at least one agent to form an agent-biomarker complex with the at least one biomarker to which it selectively binds;
detecting a level of agent-biomarker complex in the sample;
comparing the level of agent-biomarker complex to at least one pre-determined reference level of agent-biomarker complex, wherein the at least one pre-determined reference level includes a first reference level from a control population of individuals who do not have and are not developing a chronic inflammation (obesity)-associated disorder, and
i) if the level of complex differs from the first pre-determined reference level, then
concluding that the subject has or is developing an chronic inflammation (obesity)-associated disease or condition; and
ii) if the level of complex is the same as the first pre-determined reference level, then
concluding that the subject does not have and/or is not developing a chronic inflammation (obesity)-associated disease or condition.

11. The method of claim 10, wherein the at least one biomarker is selected from the group consisting of: one or more proteolytic fragments generated by cleavage of insulin-like growth factor-binding protein 3 (IGFBP-3) by a neutrophil protease; IGFBP-3; at least one neutrophil protease.

12. The method of claim 11, wherein said biomarker is IGFBP-3.

13. The method of claim 11, wherein i) levels of IGFBP-3 greater than 4500 ng/ml are considered normal; ii) levels of IGFBP-3 greater than 4100 ng/ml but less than 4500 ng/ml indicate early stage disease; and iii) levels of IGFBP-3 less than 4100 ng/ml indicate the presence of disease.

14. The method of claim 11, wherein the biomarker is at least one neutrophil protease.

15. The method of claim 14, wherein said at least one neutrophil protease is selected from the group consisting of proteinase 3 (PR3), neutrophil elastase (NE) and cathepsin G (CG).

16. The method of claim 11, wherein the biomarker is one or more proteolytic fragments.

17. The method of claim 16, wherein said one or more proteolytic fragments is selected from the group consisting of: a 28-kDa fragment formed by proteolysis of IGFBP-3 by PR-3, a 20-kDa fragment formed by proteolysis of IGFBP-3 by PR-3, a 25-kDa fragment by proteolysis of IGFBP-3 by NE, a 28-kDa fragment formed by proteolysis of IGFBP-3 by CG, a 27-kDa fragment formed by proteolysis of IGFBP-3 by CG, a 25-kDa fragment formed by proteolysis of IGFBP-3 by CG, a 20-kDa fragment formed by proteolysis of IGFBP-3 by CG, and a 18-kDa fragment formed by proteolysis of IGFBP-3 by CG.

18. The method of claim 10, wherein the chronic inflammation (obesity)-associated disorder is selected from the group consisting of: insulin resistance, type-2 diabetes, cancer and a metabolic disorder.

19. The method of claim 18, wherein said cancer is colon cancer.

20. The method of claim 10, wherein the at least one pre-determined reference level further includes a reference value selected from the group consisting of: a reference value from a control population of individuals who are developing an chronic inflammation (obesity)-associated disorder; a reference value from a control population of individuals who have an chronic inflammation (obesity)-associated disorder; a reference value from a control population of individuals who are receiving therapy to prevent an chronic inflammation (obesity)-associated disorder; a reference value from a control population of individuals who are receiving therapy to treat an chronic inflammation (obesity)-associated disorder; a reference value from the subject prior to receiving therapy to prevent an chronic inflammation (obesity)-associated disorder; a reference value from the subject prior during therapy to prevent an chronic inflammation (obesity)-associated disorder; a reference value from the subject prior to receiving therapy to prevent an chronic inflammation (obesity)-associated disorder; and a reference value from the subject during therapy to treat an chronic inflammation (obesity)-associated disorder.

21. A method of preventing or treating an chronic inflammation (obesity)-associated disorder in a subject in need thereof, comprising
administering to the subject a therapeutically effective amount of one or more inhibitors that inhibit proteolysis of IGFBP-3.

22. The method of claim 21, wherein the one or more inhibitors include at least one of: an inhibitor of proteinase 3 (PR3), an inhibitor of neutrophil elastase (NE), and an inhibitor of cathepsin G (CG).

23. The method of claim 21, wherein the inhibitor of PR3 is a peptide having the amino acid sequence: LIRCAML (SEQ ID NO: 1), or derivatives or mimetics thereof.

24. An isolated peptide having the amino acid sequence LIRCAML (SEQ ID NO: 1), or an amino acid sequence that is at least 95% identical to LIRCAML (SEQ ID NO: 1).


SEQUENCE LISTING (3 sequences)

SEQ ID NO: 1 - protein, 7 aa, Artificial Sequence
  Synthetic peptide inhibitor
LIRCAML

SEQ ID NO: 2 - protein, 8 aa, Artificial Sequence
  Synthetic peptide binding site
NTGSSFVI

SEQ ID NO: 3 - protein, 8 aa, Artificial Sequence
  Synthetic peptide binding site
NTGSSFVR